(19) United States
(12) Patent Application Publication
(10) Pub. No.: US 2025/0170395 A1
(43) Pub. Date: May 29, 2025

(54) HYDROGEL CONDUCTIVITY IMPACTS SKIN DOSE FROM TUMOR TREATING FIELDS

(71) Applicant: Rhode Island Hospital, Providence, RI (US)

(72) Inventors: Eric T. WONG, Milton, MA (US); Edwin LOK, Braintree, MA (US)

(21) Appl. No.: 18/842,001

(22) PCT Filed: Feb. 27, 2023
(86) PCT No.: PCT/US2023/063368

§ 371 Date: Aug. 27, 2024

Related U.S. Application Data
(60) Provisional application No. 63/314,850, filed on Feb. 28, 2022.

(51) Int. Cl. A61N 1/36; A61N 1/04; A61N 1/06

(52) U.S. Cl. CPC A61N 1/36002; A61N 1/0408; A61N 1/0476; A61N 1/06; A61N 1/36034

(57) ABSTRACT

Described herein are compositions and methods directed to modulating and refining dosages of tumor treating fields (TTFields) at focused locations during treatment of a subject in need thereof. The methods include tuning the conductivity of a formulation located between a transducer of the TTFields device and a tumor so that the TTFields are focused in dosage applied to the tumor and less so to the skin or other sensitive areas of the subject in need. The formulations can be applied between a TTFields transducer and the skin, to at least a portion of the whole skin (e.g., skin-penetrating), as a subcutaneous formulation, or in a combined formulation.

### CROSS REFERENCE TO RELATED APPLICATIONS

This application claims benefit from U.S. Provisional Patent Application Ser. No. 63/314,850, filed Feb. 28, 2022, which is incorporated by reference in its entirety.

### FIELD OF THE INVENTION

The embodiments of the present invention relate to use of new compositions and novel methods directed to modulating and refining tumor treating fields' dosages at various locations (e.g., at skin, at tumor) during treatment of a subject in need thereof.

### BACKGROUND OF THE INVENTION

Low intensity alternating electric fields are increasingly used to treat cancers, including brain tumors such as glioblastoma tumors, ovarian cancers, and lung cancers. This type of electromagnetic field therapy is called tumor treating fields (TTFields), which can be tuned to a frequency in the range between about 100 kHz to about 500 KHz or in the range from about 100 kHz to about 300 KHz. The alternating electric fields can disrupt cell division in cancer cells by inhibiting or reducing formation of intracellular protein structures or other structures. TTFields is a therapy that can provide synergistic effects when combined with other cancer therapies. Some exemplary TTField-generating devices are manufactured by the company Novocure®. These devices are approved in the United States and Europe for the treatment of newly diagnosed and recurrent glioblastoma multiforme (GBM) and are undergoing clinical trials for several other tumor types.

A specific TTFields device, manufactured under the trade name Optune® (formerly NovoTTF-100A or currently NovoTTF-200A), is approved in the United States, Canada, Japan, Israel, and multiple countries in Europe for the treatment of newly diagnosed and recurrent glioblastoma. The devices can be used in conjunction with other regular patterns of care for patients and can be deployed in active lifestyle settings, but are only available in certain treatment centers, and require specific training and certification on the part of the prescribing physician. When a TTFields device is used on the head, electrodes resembling a kind of head covering are placed onto a patient's shaved scalp. When not in use, the device's batteries are plugged into a power outlet to be re-charged. While TTFields is undergoing clinical trials, the adverse effects of TTFields in published trials to date have included topical skin rashes caused by prolonged electrode use, not only on the scalp but on other bodily treatment areas.

Accordingly, there is a need for methods to modulate and to improve the focus of the TTFields not only on tumor treatment areas but also topical skin.

### BRIEF SUMMARY OF THE INVENTION

The embodiments of the present invention provide modulation of TTFields delivery for the treatment of cancer by tuning the conductivity of a hydrogel or other composition at the transducer-scalp interface, transducer-skin interface, at a whole skin location, or at a deeper bodily location such as a subcutaneous location, a tumor location, or a necrotic core location. It has been discovered herein that optimizing these aspects of TTFields delivery can increase tumor dosage control while minimizing toxicity at the skull, scalp, or skin.

According to an aspect of the present innovation, a method is provided. The method can include modulating with conductivity of a composition, optionally based on at least one image of a subject's body that includes information for a plurality of structures including at least one tumor positioned within the subject, one or more aspects of TTFields. The modulation can be a tuning using a conductivity of a composition. The modulation can change at least one electric field distribution applied to the subject. The at least one electric field distribution indicates an amount of electric field at individual regions of the subject that can include at least one tumor.

According to an aspect of the present innovation, a formulation is provided. The formulation can include the composition. The formulation can be applied between a transducer of a TTFields generating device and a subject's skin, can be applied on the subject's skin (e.g., similar to a sunscreen), can be applied subcutaneously to a subject's skin, can be applied as a partial penetrating skin formulation, or a combination of these applications can be provided by the formulation. The formulation can be utilized to modulate or change at least one electric field distribution from a TTField applied to the subject.

A tumor may include a necrotic core within the tumor and/or edema adjacent to the tumor. Various structures considered within a subject can include a skin surface, hair follicles, a skin depth or whole skin, a subcutaneous skin area, a resection cavity or a surgical cavity, a thickness of cerebrospinal fluid, a volume of at least one structure, for example, of scalp, white matter, grey matter, and cerebrospinal fluid. Interestingly, the technology can provide modulation of TTFields at various depths, structures, and positions, along with providing diminished side effects.

According to some aspects, modulation of TTFields at various locations can be measured by Plan Quality Metrics (PQM). The metrics can include, in some examples, generating an electric field-volume histogram (EVH), a current density volume histogram (CDVH), area under the curve in the EVH (EAUC), area under the curve (CDAUC) in the CDVH, area under the curve (SARAUC) in the SARVH, other indications of strength of the electric fields and current densities of the alternating electric fields, or a combination thereof.

In some embodiments, the present invention provides a method for modulating tumor treating fields (TTFields) for the treatment of cancer, the method comprising the steps of:
(1) obtaining a topical agent disposed between a TTField transducer and a subject's skin; the agent operative to provide a transducer-scalp or a transducer-skin interface between the transducer, agent, and/or the skin; and
(2) optimizing a conductivity of the agent to increase the penetrating dose of TTFields to the cancer and/or to minimize hotspots or toxicity at the skull, scalp, or skin interface.

The present innovation, in some embodiments, provides the above method wherein the optimizing minimizes TTField's intensity (charge carrier density) on the skull, scalp, or skin while maximizing field intensity (charge carrier density) on a tumor. In some embodiments, the method further comprises measuring a dose of TTFields at the skull, scalp, or skin interface and/or at the tumor, and/or necrotic core and/or edema; and wherein the measuring is performed before or after step 2. In some embodiments, the optimizing is performed by changing at least a portion of the agent to a different agent with a different conductivity.

In some embodiments, the agent comprises a composition of a hydrogel, organogel, suspension, cream, emollient, paste, lotion, micro or nanoparticles, or a combination thereof.

In some embodiments, the agent is in the form of a hydrogel removably affixed to the TTField transducer. The hydrogel can be configured to be peeled off by a healthcare provider such that a different hydrogel can be applied to the transducer. In some embodiments, a transducer is implanted to a location under the skin of a subject, and a conductivity is adjusted with the transducer positioned as an implant, either subcutaneously or at a deeper location.

In some embodiments, a transducer-scalp interface is provided, and a conductivity of the agent is in the range from about 0.01 S/m to about 10 S/m or wherein a transducer-skin interface is provided, and a conductivity of the agent is in the range from about 0.001 S/m to about 100 S/m.

In some embodiments, the method can further comprise applying a vascular endothelial growth factor (VEGF) inhibitor at or under the interface. According to some aspects, the inhibitor can comprise bevacizumab or a biosimilar, a VEGF tyrosine kinase small molecule inhibitor, sunitinib, sorafenib, vandetanib, or a combination thereof.

In some embodiments, the optimized agent is in place for a time period in the range from about 18 hours or more per day continuously.

In some embodiments, the topical agent is operative to penetrate at least a portion of the skin and to change the conductivity of the skin to a value in the range from about 0.001 S/m to about 100 S/m. In some embodiments, the agent can include an enhancing agent including a metal, a metal salt, or a metal oxide, for example, comprising titanium dioxide, zinc oxide, or a combination thereof. In some embodiments, the enhancing agent can be a conductivity enhancing agent, an ultraviolet light absorbing agent, or a dye (e.g., conjugated double bonds operative to absorb in a visible region). In some embodiments, the agent can include titanium dioxide, zinc oxide, menthyl anthranilate, octocrylene, octyl salicylate, oxybenzone, padimate O, ecamsule, cinoxate, phenylbenzimidazole, sulisobenzone, homosalate, dioxybenzone, avobenzone, or a combination thereof. In some embodiments, the agent can include poly (vinyl alcohol)/polyethylene glycol/graphene oxide, hyaluronic acid, dimethyl sulfoxide, PEO (Polyethylene-Oxide)/PVP (polyvinylpyrrolidone), polysaccharide (natural), gum karaya (natural), polyacrylamide (synthetic polymer), alginate, or a combination of the above. In some embodiments, the composition is non-hydrophilic, latex free, hypoallergenic, or a combination thereof. In some embodiments, the composition further comprises an additive, an anti-vascular endothelial growth factor, a vitamin, an osmolality adjusting agent, a penetrating agent, or a combination thereof. Osmolality adjusting agents are known in the art and can include an inert solute such as a sugar or a salt.

In some embodiments, a method for modulating tumor treating fields (TTFields) for the treatment of cancer is provided, the method comprising the steps of:
(1) administering a subcutaneous agent to an area disposed under a subject's skin, which is further under a TTField transducer; the agent operative to provide a conductivity under a transducer-scalp or a transducer-skin interface between the transducer, agent, and/or skin; and
(2) optimizing a conductivity of the agent to increase the penetrating dose of TTFields to the cancer and/or to minimize hotspots or toxicity at the skull, scalp, or skin interface.

In some embodiments, a subcutaneous agent can include an osmolality of the agent in the range from about 50 mOsm/kg to about 600 mOsm/kg, or about 50 mOsm/kg to about 500 mOsm/kg, optionally at about 300 mOsm/kg. While other ranges could be used, these ranges can reduce pain or discomfort in a subject.¹

In some embodiments, the method of use of a subcutaneous agent can further comprise measuring a dose of TTFields at the skull, scalp, or skin interface and/or at the tumor; and wherein the measuring is performed before or after step 2. In some embodiments, the optimizing is performed by changing at least a portion of the agent to a different agent with a different conductivity.

In some embodiments, the conductivity of a subcutaneous agent is provided such that a transducer-scalp and/or a transducer-skin interface is provided and a conductivity of the agent is in the range from about 0.001 S/m to about 100 S/m.

In some embodiments, the method further comprises applying a vascular endothelial growth factor (VEGF) inhibitor at or under the interface. The inhibitor can optionally comprise bevacizumab or a biosimilar, a VEGF tyrosine kinase small molecule inhibitor, sunitinib, sorafenib, vandetanib, or a combination thereof.

In the various embodiments, the electric field is an alternating electric field at a frequency in the range from about 100 kHz to about 500 kHz, or in the range from about 100 kHz to about 300 kHz,² or at about 150 kHz, or optionally at about 200 kHz. In some embodiments the frequency can be about 150 kHz for body or about 200 kHz for head treatments.

In some embodiments, the methods disclosed herein are used in combination with a surgery, a surgical procedure including entering the skull, chemotherapy, targeted therapy, radiotherapy, and/or immunotherapy. In some embodiments, the methods disclosed herein are used in combination with an antibiotic, anti-inflammatory, corticosteroid, anti-allergen or hypoallergenic composition, a skin barrier, or a combination thereof.

In some embodiments, a method of investigating, diagnosing, and/or treating a disease or condition is disclosed herein, comprising any of the previously described methods. In some embodiments, a method for designing a transducer array for delivering TTFields or a device, comprising any of the methods disclosed herein is provided. In some embodiments, a method for minimizing side effects on a skin surface of a subject under administration of TTFields is provided, the method including any of the previously described methods.

In some embodiments, a kit for optimizing an application of TTFields comprising instructions including the methods disclosed herein is provided; optionally wherein the kit comprises one or more of a selection of agents with different conductivities for use with the instructions.

In some embodiments, a method of making a formulation for optimization of application of TTFields, the method comprising optimizing the formulation for a conductivity as described in any of the previously described methods. In some embodiments, the method of making can further comprise electron beam curing, thermo-reactive curing, UV curing, freeze thawing, dehydration, solvent exchange(s), rehydration, addition of particles or nanoparticles, or a combination thereof.

In some embodiments, a conductive composition for modulating tumor treating fields (TTFields) is disclosed herein, the composition comprising a gel or thickener including a conductivity; optionally wherein the composition is made by the methods described above. In some embodiments, the composition can be wherein the gel comprises a hydrogel, organogel, suspension, cream, emollient, paste, lotion, micro or nanoparticles, or a combination thereof. In some embodiments, the composition can be configured wherein the composition comprises poly (vinyl alcohol)/polyethylene glycol/graphene oxide, hyaluronic acid, dimethyl sulfoxide, PEO (Polyethylene-Oxide)/PVP (polyvinylpyrrolidone), polysaccharide (natural), gum karaya (natural), polyacrylamide (synthetic polymer), alginate, titanium dioxide, zinc oxide, or a combination thereof. In some embodiments, the composition is non-hydrophilic, latex free, hypoallergenic, or a combination thereof. In some embodiments, the composition further comprises an additive, an anti-vascular endothelial growth factor, a vitamin, an osmolality adjusting agent, a penetrating agent, or a combination thereof. Osmolality adjusting agents are known in the art and can include an inert solute such as a sugar or a salt.

In some embodiments, a formulation disclosed herein can include a penetrating agent comprising a vesicular carrier, a sulfoxide, azone, urea, a fatty acid, an alcohol, or a glycol. As used above, the term “agent” can refer to an active ingredient, a composition, a formulation, or a mixture. The methods can be provided with a system, computer software, and patient specific implementation. Other implementations are also described and recited herein.

### BRIEF DESCRIPTION OF THE DRAWINGS

For the purpose of illustration, certain embodiments of the present invention are shown in the drawings described below. It should be understood, however, that the invention is not limited to the precise arrangements, data, dimensions, and illustrations shown. In the drawings:

FIG. 1 depicts an electric field map with relative intensity shading overlayed for example tumor data for patients' head MRIs for brain cases (FIG. 1A), thoracic CT for lung cases (FIG. 1B), and abdomen and pelvis CT for ovarian cases (FIG. 1C).

FIG. 2 provides differences in the hydrogel conductivity saturation characteristics in the tumor targets (GTV and CTV) between head and body (thorax and pelvis) models according to 95% coverage metrics. For GTV in the head models, increasing hydrogel electric conductivity from 0.1 to 0.5 S/m increased E₉₅% (FIG. 2A), SAR₉₅% (FIG. 2B), and CD₉₅% (FIG. 2C) but no further increase was noted beyond 0.5 S/m. For CTV in the thorax and pelvis models, increasing hydrogel electric conductivity from 0.001 to 10 S/m increased E₉₅% (FIG. 2D), SAR₉₅% (FIG. 2E), and CD₉₅% (FIG. 2F) but no further increase was noted beyond 10 S/m.

FIG. 3 provides differences in the hydrogel conductivity saturation characteristics in the tumor targets (GTV and CTV) between head and body (thorax and pelvis) models according to median or 50% coverage metrics. For GTV in the head models, increasing hydrogel electric conductivity from 0.1 to 0.5 S/m increased hotspots E₅₀% (FIG. 3A), SAR₅₀% (FIG. 3B), and CD₅₀% (FIG. 3C) but no further increase was noted beyond 0.5 S/m. For CTV in the thorax and pelvis models, increasing hydrogel electric conductivity from 0.001 to 10 S/m increased hotspots E₅₀% (FIG. 3D), SAR₅₀% (FIG. 3E), and CD₅₀% (FIG. 3F) but no further increase was noted beyond 10 S/m.

FIG. 4 provides differences in the hydrogel conductivity saturation characteristics in the tumor targets (GTV and CTV) between head and body (thorax and pelvis) models according to hotspots or 5% coverage metrics. For GTV in the head models, increasing hydrogel electric conductivity from 0.1 to 0.5 S/m increased hotspots E₅% (FIG. 4A), SAR₅% (FIG. 4B), and CD₅% (FIG. 4C) but no further increase was noted beyond 0.5 S/m. For CTV in the thorax and pelvis models, increasing hydrogel electric conductivity from 0.1 to 10 S/m increased hotspots E₅% (FIG. 4D), SAR₅% (FIG. 4E), and CD₅% (FIG. 4F) but no further increase was noted beyond 10 S/m.

FIG. 5 provides differences in the hydrogel conductivity saturation characteristics between the scalp in the head and skin in the thorax and pelvis models according to hotspots or 5% coverage metrics. For scalp in the head models, increasing hydrogel electric conductivity from 0.1 to 0.5 S/m increased hotspots E₅% (FIG. 5A), SAR₅% (FIG. 5B), and CD₅% (FIG. 5C) but no further increase was noted beyond 0.5 S/m. For skin in the thorax and pelvis models, increasing hydrogel electric conductivity from 0.1 to 10 S/m increased hotspots E₅% (FIG. 5D), SAR₅% (FIG. 5E), and CD₅% (FIG. 5F) but no further increase was noted beyond 10 S/m.

FIG. 6 provides differences in the hydrogel conductivity saturation characteristics between the scalp in the head and the skin in the thorax and pelvis models according to 95% coverage metrics. For scalp in the head models, increasing hydrogel electric conductivity from 0.1 to 0.5 S/m increased E₉₅% (FIG. 6A), SAR₉₅% (FIG. 6B), and CD₉₅% (FIG. 6C) but no further increase was noted beyond 0.5 S/m. For skin in the thorax and pelvis models, increasing hydrogel electric conductivity from 0.1 to 10 S/m increased E₉₅% (FIG. 6D), SAR₉₅% (FIG. 6E), and CD₉₅% (FIG. 6F) but no further increase was noted beyond 10 S/m.

FIG. 7 provides differences in the hydrogel conductivity saturation characteristics between the scalp in the head and the skin in the thorax and pelvis models according to median or 50% coverage metrics. For scalp in the head models, increasing hydrogel electric conductivity from 0.1 to 0.5 S/m increased E₅₀% (FIG. 7A), SAR₅₀% (FIG. 7B), and CD₅₀% (FIG. 7C) but no further increase was noted beyond 0.5 S/m. For skin in the thorax and pelvis models, increasing hydrogel electric conductivity from 0.1 to 10 S/m increased E₅₀% (FIG. 7D), SAR₅₀% (FIG. 7E), and CD₅₀% (FIG. 7F) but no further increase was noted beyond 10 S/m.

FIG. 8 provides electric fields strength (E), as a function of modulating electric conductivity of the whole skin surface for head/brain, received by 95%, 50% (median), and 5% (hotspots) volume for GTV E₉₅% (FIG. 8A), GTV E₅₀% (FIG. 8B), GTV E₅% (FIG. 8C), Necrotic Core E₉₅% (FIG. 8D), Necrotic Core E₅₀% (FIG. 8E), Necrotic Core E₅% (FIG. 8F), GTV/CTV E₉₅% (FIG. 8G), GTV/CTV E₅₀% (FIG. 8H), and GTV/CTV E₅% (FIG. 8I). FIGS. 8A-8F are for brain areas, and FIGS. 8G-8I are for body areas.

FIG. 9 provides electric fields strength (E), as a function of modulating electric conductivity of the whole skin scalp surface, for head areas received by 95%, 50% (median), and 5% (hotspots) volume for scalp E₉₅% (FIG. 9A), scalp E₅₀% (FIG. 9B), scalp E₅% (FIG. 9C), skin E₉₅% (FIG. 9D), skin E₅₀% (FIG. 9E), skin E₅% (FIG. 9F), skull E₉₅% (FIG. 9G), skull E₅₀% (FIG. 9H), and skull E₅% (FIG. 9I).

FIG. 10 provides current density (CD), as a function of modulating electric conductivity of the whole skin surface for head/brain, received by 95%, 50% (median), and 5% (hotspots) volume for GTV CD₉₅% (FIG. 10A), GTV CD₅₀% (FIG. 10B), GTV CD₅% (FIG. 10C), Necrotic Core CD₉₅% (FIG. 10D), Necrotic Core CD₅₀% (FIG. 10E), Necrotic Core CD₅% (FIG. 10F), GTV/CTV CD₉₅% (FIG. 10G), GTV/CTV CD₅₀% (FIG. 10H), and GTV/CTV CD₅% (FIG. 10I). FIGS. 10A-10F are for brain areas, and FIGS. 10G-10I are for body areas.

FIG. 11 provides current density (CD), as a function of modulating electric conductivity of the whole scalp surface, for head areas received by 95%, 50% (median), and 5% (hotspots) volume for scalp CD₉₅% (FIG. 11A), scalp CD₅₀% (FIG. 11B), scalp CD₅% (FIG. 11C), skin CD₉₅% (FIG. 11D), skin CD₅₀% (FIG. 11E), skin CD₅% (FIG. 11F), skull CD₉₅% (FIG. 11G), skull CD₅₀% (FIG. 11H), and skull CD₅% (FIG. 11I).

FIG. 12 provides specific absorption rate (SAR), as a function of modulating electric conductivity of the whole skin surface for head/brain, received by 95%, 50% (median), and 5% (hotspots) volume for GTV SAR₉₅% (FIG. 12A), GTV SAR₅₀% (FIG. 12B), GTV SAR₅% (FIG. 12C), Necrotic Core SAR₉₅% (FIG. 12D), Necrotic Core SAR₅₀% (FIG. 12E), Necrotic Core SAR₅% (FIG. 12F), GTV/CTV SAR₉₅% (FIG. 12G), GTV/CTV SAR₅₀% (FIG. 12H), and GTV/CTV SAR₅% (FIG. 12I). FIGS. 12A-12F are for brain areas, and FIGS. 12G-12I are for body areas.

FIG. 13 provides specific absorption rate (SAR), as a function of modulating electric conductivity of the whole scalp surface, for head areas received by 95%, 50% (median), and 5% (hotspots) volume for scalp SAR₉₅% (FIG. 13A), scalp SAR₅₀% (FIG. 13B), scalp SAR₅% (FIG. 13C), skin SAR₉₅% (FIG. 13D), skin SAR₅₀% (FIG. 13E), skin SAR₅% (FIG. 13F), skull SAR₉₅% (FIG. 13G), skull SAR₅₀% (FIG. 13H), and skull SAR₅% (FIG. 13I).

FIG. 14 provides electric fields strength (E), with function of varying electric conductivity of a subcutaneous formulation (e.g., injected formulation), received by 95%, 50% (median), and 5% (hotspots) volume for GTV E₉₅% (FIG. 14A), GTV E₅₀% (FIG. 14B), GTV E₅% (FIG. 14C), Necrotic Core E₉₅% (FIG. 14D), Necrotic Core E₅₀% (FIG. 14E), and Necrotic Core E₅% (FIG. 14F).

FIG. 15 provides electric fields strength (E), with function of varying electric conductivity of a subcutaneous formulation (e.g., injected formulation) for head areas (e.g., scalp/skull) received by 95%, 50% (median), and 5% (hotspots) volume for scalp E₉₅% (FIG. 15A), scalp E₅₀% (FIG. 15B), scalp E₅% (FIG. 15C), skull E₉₅% (FIG. 15D), skull E₅₀% (FIG. 15E), and skull E₅% (FIG. 15F).

### DETAILED DESCRIPTION OF THE INVENTION

The subject innovation is now described, in some examples with reference to the drawings, wherein like references can be used to refer to like elements throughout. In the following description, for purposes of explanation, numerous specific details are set forth in order to provide a thorough understanding of the present invention. It may be evident, however, that the present invention may be practiced without these specific details. It is to be appreciated that certain aspects, modes, embodiments, variations and features of the invention are described below in various levels of detail in order to provide a substantial understanding of the present invention.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

As used in this specification and the appended claims, the singular forms “a,” “an” and “the” include plural referents unless the content clearly dictates otherwise. For example, reference to “a cell” includes a combination of two or more cells, and the like.

As used herein, the term “approximately” or “about” in reference to a value or parameter are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value). As used herein, reference to “approximately” or “about” a value or parameter includes (and describes) embodiments that are directed to that value or parameter. For example, description referring to “about X” includes description of “X”.

As used herein, the term “or” means “and/or.” The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The term “statistically significant” or “significantly” refers to statistical significance and generally means a two standard deviation (2SD) or greater difference.

As used herein, the term “subject” refers to a mammal, including but not limited to a dog, cat, horse, cow, pig, sheep, goat, chicken, rodent, or primate. Subjects can be house pets (e.g., dogs, cats), agricultural stock animals (e.g., cows, horses, pigs, chickens, etc.), laboratory animals (e.g., mice, rats, rabbits, etc.), but are not so limited. Subjects include human subjects. The human subject may be a pediatric, adult, or a geriatric subject. The human subject may be of either sex.

As used herein, the terms “effective amount” and “therapeutically effective amount” include an amount sufficient to modulate a treatment, or prevent or ameliorate a manifestation of disease or medical condition, such as cancer. It will be appreciated that there will be many ways known in the art to determine the effective amount for a given application. For example, the pharmacological methods for dosage determination may be used in the therapeutic context. In the context of therapeutic or prophylactic applications, the amount of a composition administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. The compositions can also be administered in combination with one or more additional therapeutic compounds.

As used herein, the terms “treat,” “treatment,” “treating,” or “amelioration” when used in reference to a disease, disorder or medical condition, refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, alleviation of one or more symptom(s), diminishment of extent of the deficit, stabilized (i.e., not worsening) state of a tumor or malignancy, delay or slowing of tumor growth and/or metastasis, and an increased lifespan as compared to that expected in the absence of treatment.

As used herein, the term “long-term” administration means that the therapeutic agent or drug is administered for a period of at least 12 weeks. The therapeutic agent or drug may refer to a formulation, composition, or agent. The formulation can be changed to a fresh formulation during administration. This includes that the therapeutic agent or drug is administered such that it is effective over, or for, a period of at least 12 weeks and does not necessarily imply that the administration itself takes place for 12 weeks, e.g., if sustained release compositions or long-acting therapeutic agent or drug is used. Thus, the subject is treated for a period of at least 12 weeks. In many cases, long-term administration is for at least 4, 5, 6, 7, 8, 9 months or more, or for at least 1, 2, 3, 5, 7 or 10 years, or more.

The administration of the compositions contemplated herein may be carried out in any convenient manner, including by application to a transducer or device that is subsequently applied to a subject, topical application, absorption, injection, ingestion, transfusion, implantation or transplantation. In an example embodiment, compositions are applied as a hydrogel. The phrases “parenteral administration” and “administered parenterally” as used herein refers to modes of administration other than enteral and topical administration, usually by injection, and includes, without limitation, intravascular, intravenous, intramuscular, intraarterial, intrathecal, intracapsular, intraorbital, intratumoral, intracardiac, intradermal, intraperitoneal, transtracheal, subcutaneous, subdermal, subcuticular, intraarticular, subcapsular, subarachnoid, intraspinal and intrasternal injection and infusion. In one contemplated embodiment, the compositions contemplated herein are administered to a subject by direct injection into a tumor, lymph node, or site of treatment.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g., the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, the term “conductivity” is a material's ability to conduct electric current. The SI unit of conductivity is siemens per metre (S/m). Resistivity and conductivity are intensive properties of materials. The term “conductivity” includes ionic conductivity or charge carrier conductivity in amorphous, semi-crystalline, liquid crystal, crystalline, and glassy materials. As such, the term “conductivity” can include ionic conduction and/or charge carrier conduction which is further described below.

As used herein, the term “cancer” relates generally to a class of diseases or conditions in which abnormal cells divide without control and can invade nearby tissues. Cancer cells can also spread to other parts of the body through the blood and lymph systems. There are several main types of cancer. Carcinoma is a cancer that begins in the skin or in tissues that line or cover internal organs. Sarcoma is a cancer that begins in bone, cartilage, fat, muscle, blood vessels, or other connective or supportive tissue. Leukemia is a cancer that starts in blood-forming tissue such as the bone marrow and causes large numbers of abnormal blood cells to be produced and enter the blood. Lymphoma and multiple myeloma are cancers that begin in the cells of the immune system. Central nervous system cancers are cancers that begin in the tissues of the brain and spinal cord. The technology disclosed herein can be applied to treatment, optimization, or modulation using TTFields applied to any “cancer”.

In some embodiments of any of the aspects, the cancer is a primary cancer. In some embodiments of any of the aspects, the cancer is a malignant cancer. As used herein, the term “malignant” refers to a cancer in which a group of tumor cells display one or more of uncontrolled growth (i.e., division beyond normal limits), invasion (i.e., intrusion on and destruction of adjacent tissues), and metastasis (i.e., spread to other locations in the body via lymph or blood). As used herein, the term “metastasize” refers to the spread of cancer from one part of the body to another. A tumor formed by cells that have spread is called a “metastatic tumor” or a “metastasis.” The metastatic tumor contains cells that are like those in the original (primary) tumor.

As used herein, the term “benign” or “non-malignant” refers to tumors that may grow larger but do not spread to other parts of the body. Benign tumors are self-limited and typically do not invade or metastasize.

A “cancer cell” or “tumor cell” refers to an individual cell of a cancerous growth or tissue. A tumor refers generally to a swelling or lesion formed by an abnormal growth of cells, which may be benign, pre-malignant, or malignant. Most cancer cells form tumors, but some, e.g., leukemia, do not necessarily form tumors. For those cancer cells that form tumors, the terms cancer (cell) and tumor (cell) are used interchangeably.

A subject that has a cancer or a tumor is a subject having objectively measurable cancer cells present in the subject's body. Included in this definition are malignant, actively proliferative cancers, as well as potentially dormant tumors or micrometastatses. Cancers which migrate from their original location and seed other vital organs can eventually lead to the death of the subject through the functional deterioration of the affected organs. Hemopoietic cancers, such as leukemia, are able to out-compete the normal hemopoietic compartments in a subject, thereby leading to hemopoietic failure (in the form of anemia, thrombocytopenia and neutropenia) ultimately causing death.

Examples of cancer include but are not limited to, carcinoma, lymphoma, blastoma, sarcoma, leukemia, basal cell carcinoma, biliary tract cancer; bladder cancer; bone cancer; brain and CNS cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma (GBM); hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); lymphoma including Hodgkin's and non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; as well as other carcinomas and sarcomas; as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome.

A “cancer cell” is a cancerous, pre-cancerous, or transformed cell, either in vivo, ex vivo, or in tissue culture, that has spontaneous or induced phenotypic changes that do not necessarily involve the uptake of new genetic material. Although transformation can arise from infection with a transforming virus and incorporation of new genomic nucleic acid, or uptake of exogenous nucleic acid, it can also arise spontaneously or following exposure to a carcinogen, thereby mutating an endogenous gene. Transformation/cancer is associated with, e.g., morphological changes, immortalization of cells, aberrant growth control, foci formation, anchorage independence, malignancy, loss of contact inhibition and density limitation of growth, growth factor or serum independence, tumor specific markers, invasiveness or metastasis, and tumor growth in suitable animal hosts such as nude mice.

A subject can be one who has been previously diagnosed with or identified as suffering from or having a condition in need of treatment (e.g., a cancer) or one or more complications related to such a condition, and optionally, but need not have already undergone treatment for a condition or the one or more complications related to the condition. Alternatively, a subject can also be one who has not been previously diagnosed as having a condition in need of treatment or one or more complications related to such a condition. For example, a subject can be one who exhibits one or more risk factors for a condition or one or more complications related to a condition or a subject who does not exhibit risk factors. A “subject in need” of treatment for a particular condition can be a subject having that condition, diagnosed as having that condition, or at risk of developing that condition.

Conductivity Impacts Skin Dose from Tumor Treating Fields

Tumor Treating Fields (TTFields) disrupt tumor cells while they are undergoing mitosis. In patients, transducer arrays are applied to scalp or body surface for treatment of glioblastomas, mesothelioma, or other systemic malignancies (e.g., cancers). Dermatologic complications are thought to be related to hydrogel, which is applied between the electrodes and scalp or skin surface to facilitate electric field penetration. But the high intensity of TTFields on these surfaces may also be a contributing factor.

TTFields therapy is standard-of-care for patients with newly diagnosed and recurrent glioblastomas.^{3,4} With some devices, its anti-cancer efficacy is derived from alternating electric fields tuned to a frequency of 200 kHz, which disrupt tumor cells in mitosis, cause double strand DNA breaks, and impose other types of cellular stress resulting in secondary autophagy or immunogenic cell death.^{5, 6, 7, 8} TTFields are delivered continuously to the patient's head via two pairs of orthogonally positioned transducer arrays.⁹ Compliance at 75% or continuous use for 18 hours or longer is an important determinant of treatment efficacy.¹⁰ Furthermore, TTFields are approved for treatment of mesothelioma and the 4 arrays are placed on the patient's chest wall. 11 Randomized clinical trials are also in the process of being conducted to determine the efficacy of TTFields for advanced stage non-small cell lung cancer (NCT02973789), locally advanced pancreatic carcinoma (NCT03377491), and ovarian carcinoma (NCT03940196), all in combination with conventional chemotherapies, targeted therapy and/or immunotherapy. Therefore, TTFields will likely be incorporated as an adjuvant treatment for a variety of malignancies in the future. The technology contemplates the incorporation of TTFields for various malignancies, while using cancer to demonstrate the advantages of the methods and compositions disclosed herein.

Despite the fewer number of adverse side effects from TTFields compared to conventional chemotherapy and radiotherapy, a major complication associated with continuous use is local scalp or skin breakdown.^{12, 13} These dermatologic complications, which include erythema, pruritis, and secondary infection,¹² are thought to be a result of allergic reactions from hydrogel, which is situated between the electrodes and scalp or skin to facilitate electric field penetration, rather than from TTFields emanating from the electrodes. High ambient temperature and hyperhidrosis are also modifying factors that may alter the severity of these complications.¹² However, if the dermatologic complications are unrelated to hydrogel allergy but associated with intensity of TTFields, then there is a possibility to optimize the delivery of TTFields by minimizing field intensity on the scalp while maximizing it on the tumor. Therefore, in an example, analysis of 12 patient models (7 head model from glioblastoma patients, 3 thorax models from non-small cell carcinoma patients, and 2 pelvis models from ovarian cancer patients) and investigation of the TTFields' coverage effects on scalp and target as well as skin and target under different hydrogel electric conductivities is presented herein. It is found that there is a general difference in the optimum conductivity between head and body (thorax and pelvis) models; but variability also exists among individual patients within each anatomic site.

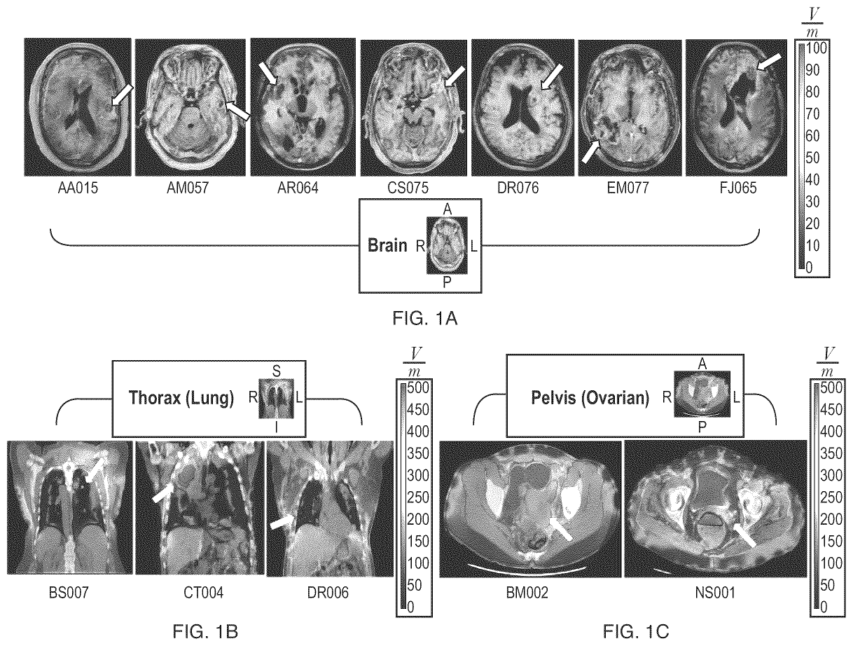

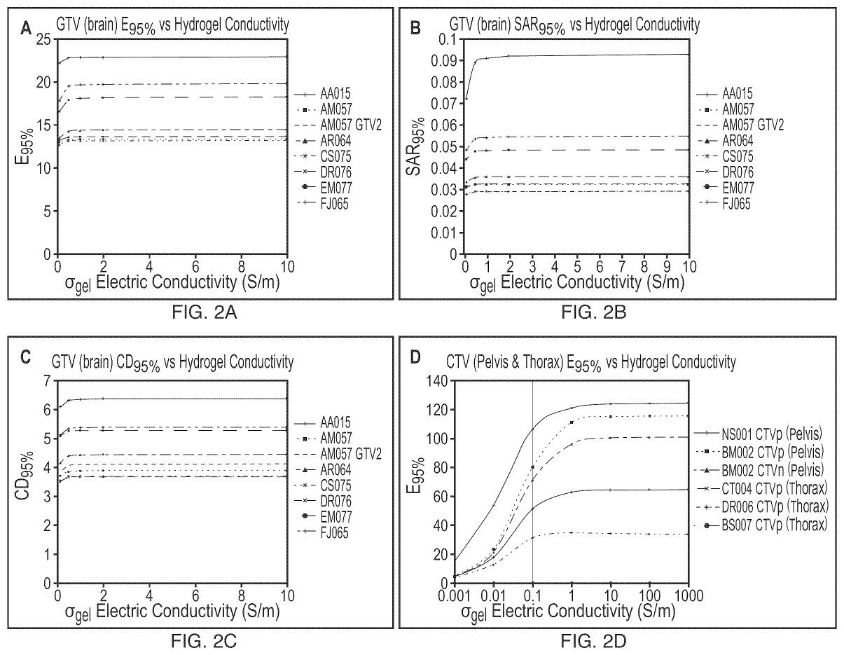

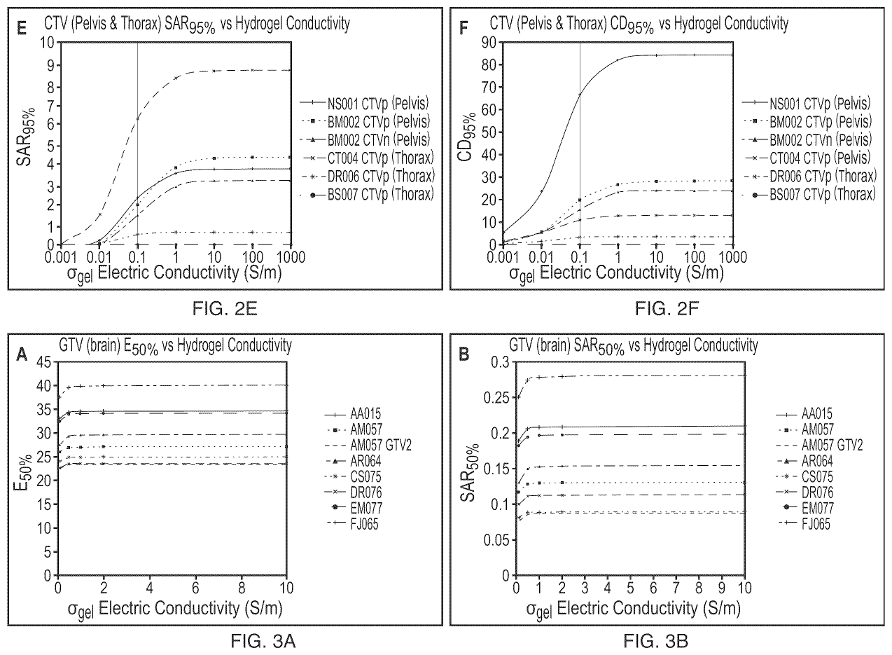

The MRI data from 7 glioblastoma patients (FIG. 1A), who underwent TTFields therapy, as well as the positron emission tomography-computed tomography (PET-CT) imaging data from 3 lung carcinoma (FIG. 1B) and 2 ovarian carcinoma patients (FIG. 1C), are used to segment normal intracranial, intrathoracic, and intrapelvic anatomic structures under an institutional review board approved protocol. T1 post-gadolinium MR sequences were used as previously described to aid the delineation of gross tumor volumes in the brain tumor models.¹⁴ PET data was used to aid the delineation of the PET-positive gross tumor volume (GTV) in the lung models, where a uniform 3 mm expansion around the GTV was performed to create the clinical target volumes (CTV). Method of target delineation for the pelvic models were similarly described in our prior publication.¹⁵ A 3-dimensional finite element mesh was then generated for each model in ScanIP (Synopsys, Mountain View, CA) and then imported into COMSOL Multiphysics 5.5 (COMSOL, Burlington, MA). Sensitivity analysis as a function of hydrogel electric conductivity was achieved by varying the electric conductivity parameter in each model in log unit intervals between 0.001 S/m through 1000 S/m. The nominal value for hydrogel electric conductivity is 0.1 S/m as previously used across multiple modeling studies.^{14, 16, 17}

Head models were not modeled with electric conductivity of the hydrogel set beyond 10 S/m due to lower potential electric field saturation, while the thorax and pelvis models employed hydrogel electric conductivity values beyond 10 S/m and up to 1000 S/m due to the assumption that a larger girth of the body would require a higher electric field penetration. Appropriate material properties and boundary conditions as described in prior studies were then specified, and magnetic fields were assumed to be negligible at the frequency range for the operation of TTFields.^{14, 15} The AC/DC module from Page 4/17 COMSOL Multiphysics was used to solve for the electric field distribution in each model. In order to quantify the distributions as a function of hydrogel conductivity, plan quality metrics (PQMs) derived from each model's respective volume histograms, were used to make comparisons within Excel 2016 (Microsoft, Redmond, WA).

PQMs included 95% coverage metrics such as E₉₅%, SAR₉₅%, and CD₉₅%, which are electric fields strength received by 95% volume of a tissue, specific absorption rate (or the rate at which energy is absorbed) received by 95% volume of a tissue, and current density received by 95% volume of a tissue, respectively. The 5% metrics such as E₅%, SAR₅%, CD₅% were used to quantify hotspots within tissues, specifically the intensity of each metric within 5% of tissue. Median coverage for electric field, SAR and current density were also utilized to quantitatively compare between models, denoted by E₅₀%, SAR₅₀%, and CD₅₀%, respectively.

Ttfields Distribution Saturates with Respect to Hydrogel Electric Conductivity

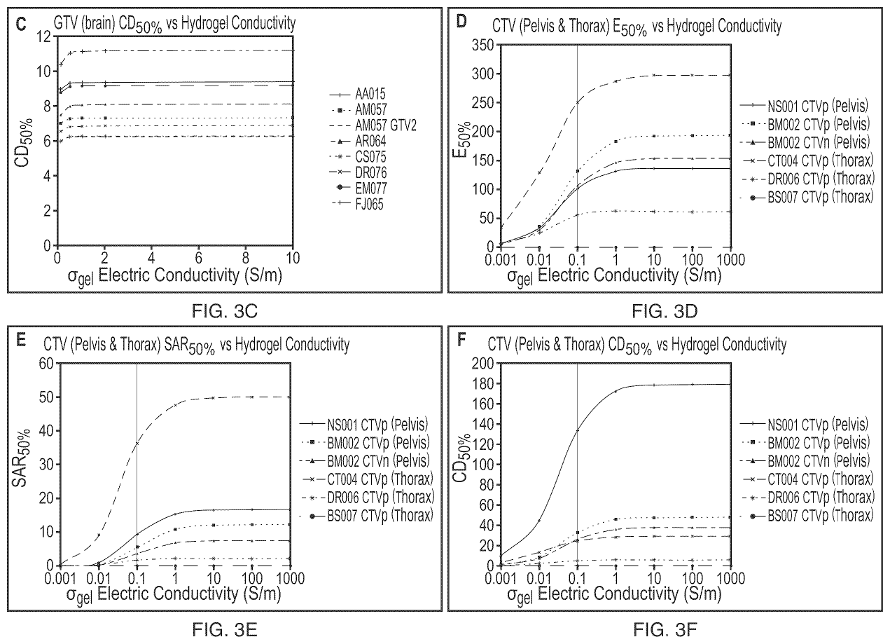

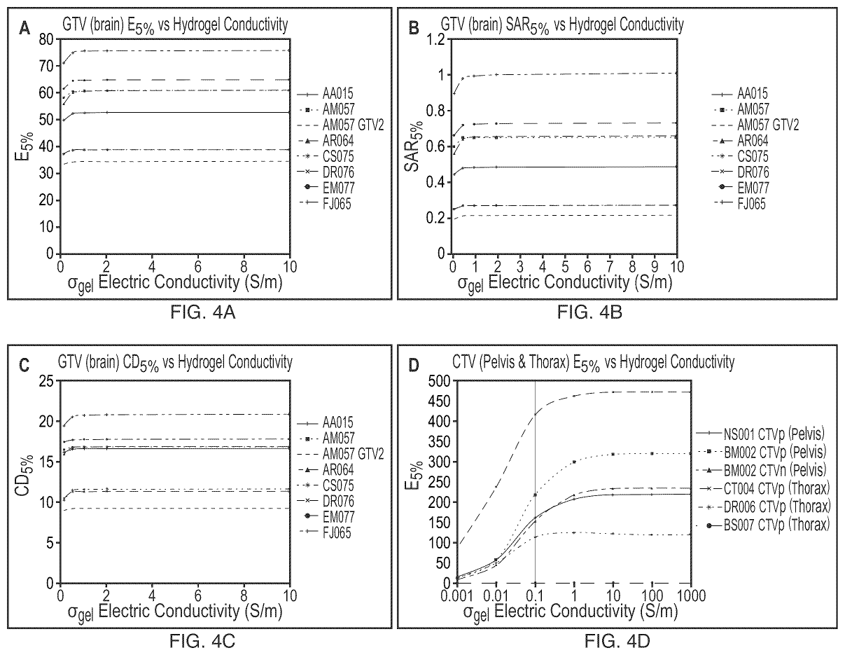

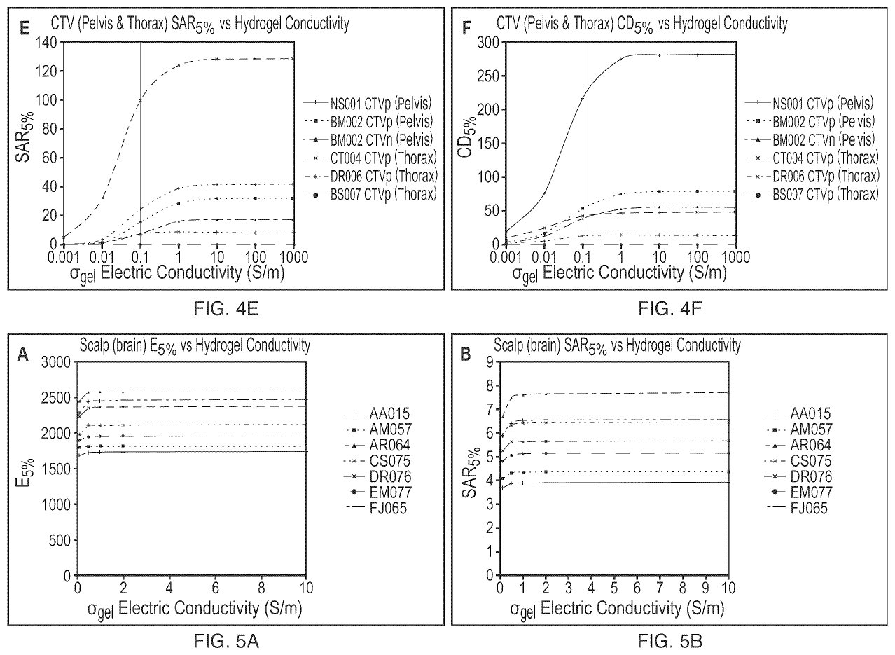

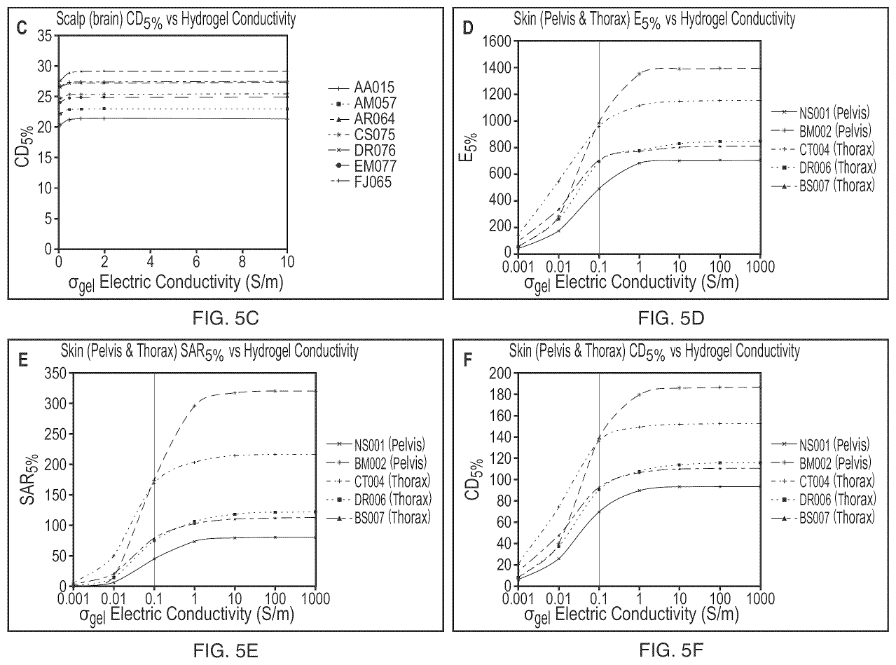

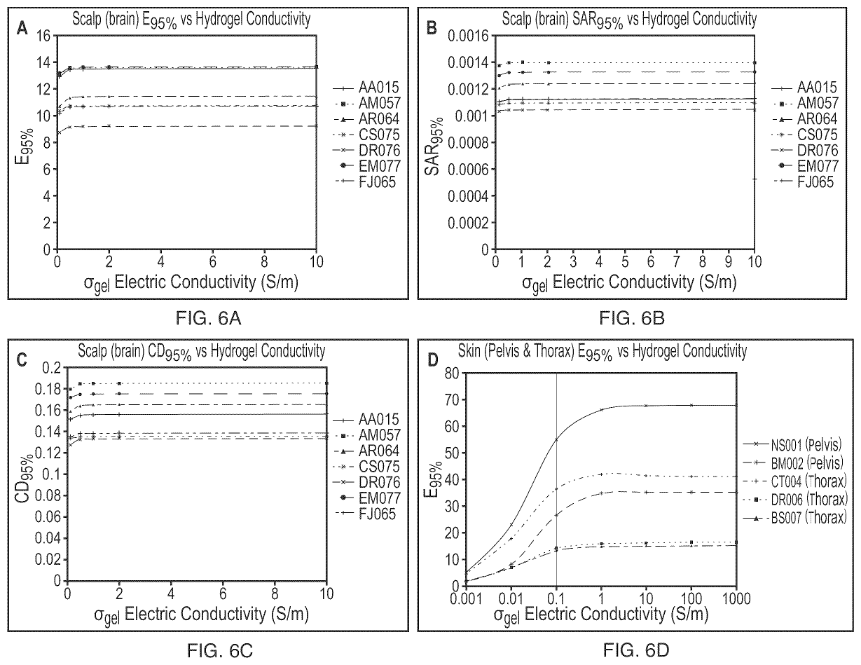

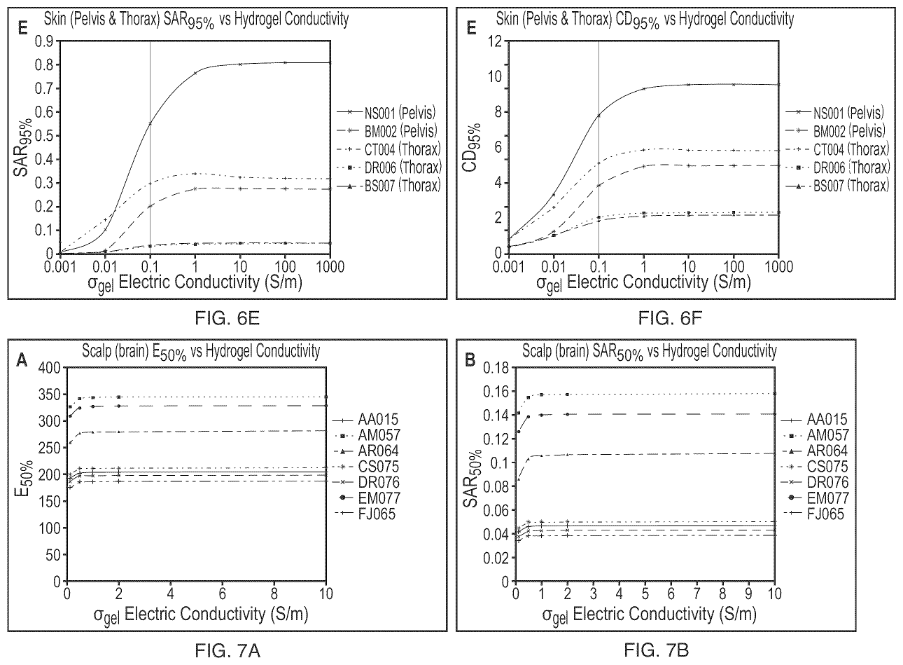

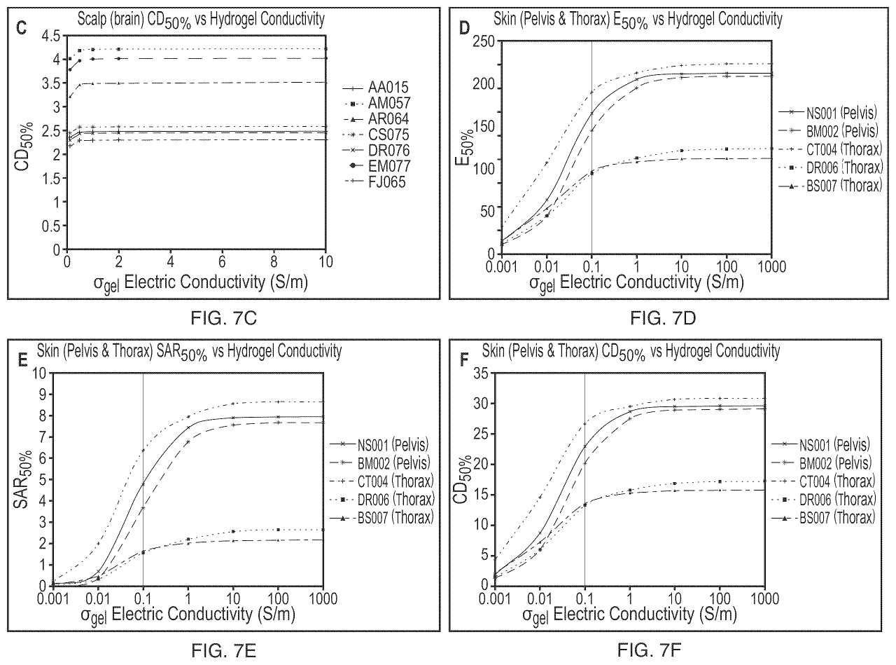

Our studies reveal that there is a saturation point at which increasing the hydrogel electric conductivity beyond a certain value results in no further increase in TTFields coverage at the GTV. This saturation point appears to be different in the head, thorax, and pelvis models (FIG. 1A, FIG. 1B, FIG. 1C). For the head models, the saturation point is near 0.5 S/m and increasing the hydrogel conductivity from 0.1 to 0.5 S/m resulted an average increase of +4.5% (range 2.0-9.4%) in E₉₅%, +8.6% (range 3.5-23.8%) in SAR₉₅%, and +4.4% (range 3.3-6.5%) in CD₉₅% at the GTV (FIGS. 2A-C). There is negligible change in 95% TTFields coverage beyond 0.5 S/m. Similarly, the median coverage at the GTV increases on average by +4.2% (range 2.9-6.7%) in E₅₀%, +10.2% (range 6.9-14.7%) in SAR₅₀%, and +4.5% (range 3.2-6.8%) in CD₅₀% (FIGS. 3A-C). The GTV hotspot, as denoted by E₅%, SAR₅%, and CD₅%, increases by an average of +4.6% (range 2.7-7.4%), +9.2% (range 7.5-14.5%), and +4.8% (range 1.7-11.4%), respectively (FIGS. 4A-C). In addition, the scalp hotspot, as denoted by E₅%, SAR₅%, and CD₅%, increases on average by +4.2% (range 0.6-7.2%), +7.5% (range 5.2-12.0%), and +3.2% (range 2.2-4.6%), respectively (FIGS. 5A-C). However, there is also negligible change in the median (50%) coverage of TTFields and hotspot (5%) beyond 0.5 S/m. The 95% and median (50%) coverage to the scalp follows a similar pattern of increase in intensity as a function of hydrogel electric conductivity and exhibited saturation points around 0.5 S/m (95%, FIGS. 6A-C and 50%, FIGS. 7A-C).

For the thorax and pelvis models, the saturation point for hydrogel conductivity is observed at 1.0 S/m and increasing the hydrogel conductivity from 0.1 to 1.0 S/m yields an average increase of +21.7% (range 2.4-41.7%) in E₉₅%, +51.3% (range 0.0-114.2%) in SAR₉₅%, and +41.0% (range 11-122.2%) in CD₉₅% at the CTV (FIGS. 2D-F). There is negligible change in 95% TTFields coverage beyond 1.0 S/m. The average increase in median coverage at the CTV, quantified by the E₅₀% metric, is +22.5% (range 3.2-38.8%) for E₅₀%, +50.4% (range 0-98.3%) for SAR₅₀%, and +24.4% (range 11.8-38.6) for CD₅₀% (FIGS. 3D-F). The CTV hotspot, as denoted by E₅%, SAR₅%, and CD₅% increases by an average of +26.6% (range 9.1-41.7%), +51.0% (range 0-114.2%), and +21.7% (range 2.4-40.7%), respectively (FIGS. 4D-F). In addition, the skin hotspot, denoted by E₅%, SAR₅%, and CD₅%, increases by +22.8% (range 9.6-38.9%), +45.7% (range 20.6-87.8%), and +20.6% (range 9.5-29.9%), respectively (FIGS. 5D-F). The 95% and median (50%) coverage to the skin also followed a similar pattern of increase in intensity as a function of hydrogel electric conductivity, although the saturation points were exhibited slightly above 1 S/m, closer to 10 S/m (FIGS. 6D-6F and FIGS. 7D-7F).

### TTFields Distribution Varies Among Different Patient Models

We observed that each patient model possesses an individualized increase in TTFields as a function of increasing hydrogel electric conductivity (e.g., FIGS. 2-7). Generally, the greatest percentage increase is observed in the scalp or skin rather than the targets GTV or CTV. For the head models (n=7, FIG. 1A), when the hydrogel electric conductivity is set at the nominal value of 0.1 S/m and experimental value of 0.5 S/m, the average increase in TTFields of the GTV is +4.7% in E₉₅%, +4.3% in E₅₀%, and +4.8% in E₅% (FIGS. 2A, 3A, and 4A). For the scalp, when the hydrogel conductivity is increased from 0.1 to 0.5 S/m, the average increase is +4.2% in E₉₅%, +5.6% in E₅₀%, and +4.4% in E₅% hotspot (FIG. 6A and FIG. 7A, FIG. 5A). In contrast, the SAR exhibits larger average increases in coverage within the GTV, at +10.7% for SAR₉₅%, +10.0% for SAR₅₀%, and +9.3% for SAR₅% (FIGS. 2B, 3B, and 4B). At the scalp, the average SAR increase for SAR₉₅%, SAR₅₀%, and SAR₅% is low at +1.3%, +12.1% and +7.8%, respectively (FIG. 6B and FIG. 7B, FIG. 5B). For current density, coverage to the GTV has average increases of +4.4% in CD₉₅%, +4.6% in CD₅₀%, and +4.4% in CD₅% hotspot when hydrogel conductivity is increased from 0.1 S/m to 0.5 S/m (FIGS. 2C, 3C, and 4C). Within the scalp, the average increases are +2.1%, +5.6% and +3.1% for CD₉₅%, CD₅₀%, and CD₅%, respectively (FIG. 6C and FIG. 7C, FIG. 5C).

A similar trend is observed in the thorax (n=3, FIG. 1B) and pelvis (n=2, FIG. 1C) models where the field intensity generally increases as a function of increasing hydrogel electric conductivity. The percentage increases in the coverage metrics were much higher than those described in the head models. Specifically, the average increases in TTFields coverage to the CTVs are +25.1%, +25.3% and +23.1% for E₉₅%, E₅₀%, and E₅%, respectively (FIGS. 2D, 3D, and 4D), while the skin experiences average increases of +19.3%, +20.9%, and +22.6% for the E₉₅%, E₅₀%, and E₅% metrics, respectively (FIGS. 6D and 7D, FIG. 5D). SAR within the CTVs increased by +51.3% in SAR₉₅%, +46.0% in SAR₅₀%, and +41.1% in SAR₅% (FIGS. 2E, 3E, and 4E). Within the skin layer of these body models, the average increase is +30.8% in SAR₉₅%, +46.2% in SAR₅₀%, and +44.5% in SAR₅% (FIGS. 6E and 7E, FIG. 5E). The CTVs have an average increase in current density also, when the hydrogel conductivity was increased from 0.1 S/m to 0.5 S/m. The CD at the CTV was increased by +28.0% in CD₉₅%, +29.3% in CD₅₀%, and +27.7% in CD₅% hotspot (FIGS. 2F, 3F, and 4F). Lastly, at the skin layer, there was an average increase of +19.7% in CD₉₅%, +20.7% in CD₅₀%, and +20.1% in CD₅% hotspot (FIGS. 6F and 7F, FIG. 5F).

### TTFields Distribution Differences Between Head and Body

We next compare the extent of changes in PQM metrics between the head and body models when the hydrogel conductivity was increased from 0.1 to 0.5 S/m (e.g., Table 1 below). For GTVs in the head models, this results in a negligible increase in standard deviation of <10% in E₉₅%, E₅₀%, E₅%, SAR₅₀%, SAR₅%, CD₉₅%, CD₅₀%, and CD₅%, except for SAR₉₅% which had an increase of 34.3%. However, for the CTVs in the thorax and pelvis models, there is a greater increase in standard deviation ranging from 13.4-32.0% across all of the PQM metrics. For scalp in the head models, increasing the hydrogel conductivity resulted in a negligible increase in standard deviation of <10% in E₉₅%, E₅₀%, SAR₉₅%, CD₉₅%, CD₅₀%, and CD₅%, except for E₅%, SAR₅₀%, and SAR₅%, which are increased by 17.4%, 10.7%, and 18.2%, respectively. In contrast, the thorax and pelvis models have a much greater increase in standard deviation ranging from 21.1% up to 54.7% across all of the PQM metrics.

### Hydrogel

Hydrogel is important for the delivery of TTFields, which is a type of alternating electric fields at 100-500 kHz, to treat cancer in the body. This hydrogel medium acts as an interface between the ceramic electrodes and the skin, which ensures the optimal transfer of charges from electrodes to the body surface. Therefore, the composition of the medium is critically important for maximizing delivery of TTFields to the treatment region(s), including, but not limited to, tumor volumes and other bodily tissues. We have shown that the medium's electrical conductivity and mass density are more important determinants than relative permittivity. Therefore, optimizing the material properties of the medium will ensure optimal delivery of Tumor Treating Fields to the target for the treatment of cancer.

The hydrogel-scalp or hydrogel-skin interface is critical for the penetration of TTFields into intracranial space or other body cavities, respectively. Kirson, et al. found that higher TTFields intensity correlated with the percent of tumor cell kill in experimental cell culture models.¹⁸ To optimize delivery of TTFields for the treatment of cancer, the penetrating dose at the GTV in the brain or CTV within the thorax or pelvis should be maximized while the hotspots on the skull, scalp, or skin surface minimized. We speculate that the conductivity of the hydrogel could alter the TTFields coverage at both GTV or CTV and the hotspot on the skull, scalp, or skin. Indeed, as the conductivity of the hydrogel increases, TTFields intensity at the GTV or CTV reached a maximum beyond which no further increase occurred, suggesting that the skull, scalp, or other skin surface may act as a sink for the applied TTFields. Therefore, there should be an optimal hydrogel conductivity that maximizes TTFields penetration into the head or body cavity while minimizing toxicities in the skull, scalp, or skin layer.

One possible explanation for the saturation characteristics may be due to the relationship between electric conductivity and the response of charge carriers under the influence of time-dependent electric fields. The concentration and allowable motion of charges are the primary drivers that determine a material's electric conductivity according to Ohm's Law. Furthermore, Gauss' Law describes the relationship between the electric field intensity and the number of charges within an enclosed space. As the conductivity of the hydrogel approaches large values, its electrical response behaves similar to that of metals, where the charge carrier density can be calculated according to the Drude model.¹⁹ Charge carrier density is usually constant for a particular material in specific states of matter and, therefore, can only conduct a maximum amount of electric charge. As we increased the electric conductivity of the hydrogel, the charge carrier density increases. However, since the electric conductivities of the neuroanatomy throughout the rest of the head models is unaltered in this study, the innate charge carrier densities also remain unchanged. Therefore, it is most likely the case that there exists such a saturation of electric field, SAR and CD intensities within the brain.

The saturation point for electric fields coverage as a function of hydrogel conductivity is twice as high for thorax and pelvis models compared to the head models. This may be due to the higher electric output from the NovoTTF-100L arrays for the thorax and pelvis²⁰ compared to the NovoTTF-100A or NovoTTF-200A for the head.²¹ Specifically, the maximum intensity for NovoTTF-100L is 1414 mA while the maximum for NovoTTF-100A or NovoTTF-200A is 900 mA, or 1.6 times higher for the body arrays. In addition, each NovoTTF-100L array has up to 20 ceramic electrodes compared to the 3×3 electrode configuration for NovoTTF-100A or NovoTTF-200A. Therefore, higher electric output combined with increased number of electrodes could contribute to the higher saturation point for hydrogel conductivity in the thorax and pelvis models.

From a biologic perspective, scalp and skin could respond by hypervascularization to counteract increasing doses of applied TTFields. Hyper-vascularized scalp or skin could shunt the electric fields away from the shallower depth of skin beneath the transducers, inducing the fields to scatter tangentially away from the point of contact rather than penetrating perpendicularly into the scalp or skin. In our prior report, scalp erythema was noted in a longitudinal fashion rather than localized just underneath the applied transducer disks.²² Compared to skin on the body, higher vascularity is necessary to support growth of hair follicles on the scalp and this notion is corroborated by transgenic mice with upregulated vascular endothelial growth factor (VEGF) have more hair compared to wild-type mice.²³ Therefore, we speculate that topical application of a monoclonal antibody against VEGF, such as bevacizumab or its biosimilar,²⁴ or VEGF tyrosine kinase small molecule inhibitors, such as sunitinib, sorafenib, and vandetanib,²⁵ may decrease scalp vascularity in response to TTFields treatment and permit a higher penetration of the fields into the scalp or skin and eventually into the intracranial or intrathoracic and intrapelvic spaces. In addition, the application of anti-VEGF drugs may attenuate the severity of erythema associated with chronic application of TTFields.

There are differences in the PQMs for electric field, SAR and CD among the individual head, thorax, and pelvis models. This type of individual variability indicates that TTFields coverage at the GTV or CTV are modulated by other parameters, including probably the geometry or location of the tumor, presence or absence of a necrotic core, association with cerebral edema, and proximity to a fluid source such as the cerebral ventricles or bladder. First, tumors possessing asymmetry and angulated geometry have higher electric field values compared to those with symmetry and less angulation.¹⁴ This is most likely from charges accumulating at the sharp corners exhibiting a higher electric force in certain regions of the tumor and therefore effecting a greater anti-tumor effect. Second, the presence of a necrotic core concentrates the electric fields to this intra-tumoral fluid-filled space and therefore enabling the GTV to accumulate a greater amount of TTFields.¹⁴ Lastly, when the tumor is adjacent to cerebral ventricle or bladder, higher charges within these fluid-filled cavities may concentrate TTFields to the respective GTV or CTV.¹⁴ Therefore, tumor-associated or organ-specific characteristics modulate the distribution of TTFields within the intracranial, intrathoracic, and intrapelvic sites.

It is concluded that TTFields delivery for the treatment of cancer can be modulated by the conductivity of the hydrogel at the transducer-scalp or transducer-skin interface. Optimizing this aspect of TTFields delivery may increase tumor control while minimizing toxicity at the skull, scalp, or skin.

### Formulations and Compositions

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as a gel, granule, lyophile for reconstitution, powder, solution, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-micro emulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation, formulation, agent, or composition) can be administered to a subject by any of a number of routes of administration including, for example, powders, granules, pastes for application; absorption through the oral mucosa (e.g., sublingually); subcutaneously; transdermally (for example as a patch applied to the skin); and topically (for example, as a cream, ointment or spray applied to the skin). In some embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

To prepare dosage forms for administration, an active ingredient can be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents.

Liquid dosage forms can include pharmaceutically acceptable gels, emulsions, lyophiles for reconstitution, micro-emulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intraocular (such as intravitreal), intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow-release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art. See, e.g., Isselbacher et al. (1996).²⁶

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In other embodiments, the active compound will be administered once daily.

The patient receiving this treatment is any animal in need, including primates, in particular humans; and other mammals such as equines bovine, porcine, sheep, feline, and canine; poultry; and pets in general.

In certain embodiments, compounds of the invention may be used alone or conjointly administered with another type of therapeutic agent.

The present disclosure includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino) ethanol, ethanolamine, ethylenediamine, N-methylglucamine, hydrabamine, 1H-imidazole, lithium, L-lysine, magnesium, 4-(2-hydroxyethyl) morpholine, piperazine, potassium, 1-(2-hydroxyethyl) pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, l-ascorbic acid, I-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, I-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, I-pyroglutamic acid, salicylic acid, sebacic acid, stearic acid, succinic acid, sulfuric acid, I-tartaric acid, thiocyanic acid, p-toluenesulfonic acid, trifluoroacetic acid, and undecylenic acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only and is not intended to limit the scope of the present invention, which is defined solely by the claims. Definitions of common terms in immunology and molecular biology can be found in The Merck Manual of Diagnosis and Therapy;²⁷ The Encyclopedia of Molecular Cell Biology and Molecular Medicine;²⁸ Molecular Biology and Biotechnology: a Comprehensive Desk Reference;²⁹ Immunology;³⁰ Janeway's Immunobiology;³¹ Lewin's Genes XI;³² Molecular Cloning: A Laboratory Manual.;³³ Basic Methods in Molecular Biology;³⁴ Laboratory Methods in Enzymology;³⁵ Current Protocols in Molecular Biology (CPMB);³⁶ Current Protocols in Protein Science (CPPS);³⁷ and Current Protocols in Immunology (CPI).³⁸

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

The methods, agents, and composition can be provided as a prodrug, which is a pharmacologically inactive medication or compound that, after intake, is metabolized (i.e., converted within the body) into an active formulation. Instead of administering an active agent directly, a corresponding prodrug can be used to improve how the drug is absorbed, distributed, metabolized, and excreted (ADME). In another example, the methods and compositions provide an agent that can change over time or can change by application of an electromagnetic radiation.

Modulating Dosages from Tumor Treating Fields

The present invention, in one of its broadest embodiments, provides a method for modulating tumor treating fields (TTFields) for the treatment of cancer, the method comprising the steps of:
(1) obtaining a topical agent disposed between a TTField transducer and a subject's skin; the agent operative to provide a transducer-scalp or a transducer-skin interface between the transducer, agent, and/or the skin; and
(2) optimizing a conductivity of the agent to increase the penetrating dose of TTFields to the cancer and/or to minimize hotspots or toxicity at the skull, scalp, or skin interface.

The present innovation, in some embodiments, provides the above method wherein the optimizing minimizes TTField's intensity (charge carrier density) on the skull, scalp, or skin while maximizing field intensity (charge carrier density) on a tumor. In some embodiments, the method further comprises measuring a dose of TTFields at the skull, scalp, or skin interface and/or at the tumor; and wherein the measuring is performed before or after step 2. In some embodiments, the optimizing is performed by changing at least a portion of the agent to a different agent with a different conductivity.

In some embodiments, the agent comprises a composition of a hydrogel, organogel, suspension, cream, emollient, paste, lotion, micro or nanoparticles, or a combination thereof.

In some embodiments, the agent is in the form of a hydrogel removably affixed to the TTField transducer. The hydrogel can be configured to be peeled off by a healthcare provider such that a different hydrogel can be applied to the transducer.

In some embodiments, a transducer-scalp interface is provided, and a conductivity of the agent is in the range from about 0.001 S/m to about 10 S/m, or in the range from about 0.1 S/m to about 5 S/m, or in the range from about 0.1 S/m to about 0.5 S/m; or wherein a transducer-skin interface is provided, and a conductivity of the agent is in the range from about 0.001 S/m to about 100 S/m, or in the range from about 0.001 S/m to about 50 S/m, or in the range from about 0.001 S/m to about 10 S/m, or in the range from about 0.001 S/m to about 1.0 S/m. In some embodiments, the conductivity of the agent is optimized in a transducer-scalp interface in the range from about 0.001 S/m to about 100 S/m, or from about 0.001 S/m to about 50 S/m, or from about 0.001 S/m to about 10 S/m, or from about 0.1 S/m to about 1 S/m, or from about 0.1 S/m to about 0.6 S/m, optionally at about 0.1 S/m, or at about 0.2 S/m, at about 0.3 S/m, at about 0.4 S/m, or at about 0.5 S/m, depending upon the specific patient and depth of treatment. In some embodiments, the transducer-skin interface is optimized when the conductivity of the agent is in the range from about 0.001 S/m to about 100 S/m, from about 0.001 S/m to about 50 S/m, from about 0.1 S/m to about 1.1 S/m, optionally at about 0.1 S/m, or at about 0.2 S/m, at about 0.3 S/m, at about 0.4 S/m, or at about 0.5 S/m, at about 0.6 S/m, at about 0.7 S/m, at about 0.8 S/m, at about 0.9 S/m, or at about 1.0 S/m, depending upon the specific patient and depth of treatment.

In some embodiments, the method can further comprise applying a vascular endothelial growth factor (VEGF) inhibitor at or under the interface. According to some aspects, the inhibitor can comprise bevacizumab or a biosimilar, a VEGF tyrosine kinase small molecule inhibitor, sunitinib, sorafenib, vandetanib, or a combination thereof.

In some embodiments, the optimized agent is in place for a time period in the range from about 18 hours or more per day, continuously.

In some embodiments, the topical agent is operative to penetrate at least a portion of the skin and to change the conductivity of the skin to a value in the range from about 0.001 S/m to about 100 S/m. In some embodiments, the conductivity of the skin is optimized at a conductivity in the range from about 0.001 S/m to about 90 S/m, or in the range from about 0.001 S/m to about 50 S/m, or in the range from about 0.1 S/m to about 11 S/m, optionally at about 1 S/m, or at about 2 S/m, at about 3 S/m, at about 4 S/m, or at about 5 S/m, at about 6 S/m, at about 7 S/m, at about 8 S/m, at about 9 S/m, or at about 10 S/m, depending upon the specific patient and depth of treatment. In some embodiments, the agent can include a conductive agent comprising titanium dioxide, zinc oxide, or a combination thereof.

In some embodiments, a method for modulating tumor treating fields (TTFields) for the treatment of cancer is provided, the method comprising the steps of:
(1) administering a subcutaneous agent to an area disposed under a subject's skin, which is further under a TTField transducer; the agent operative to provide a conductivity under a transducer-scalp or a transducer-skin interface between the transducer, agent, and/or skin; and
(2) optimizing a conductivity of the agent to increase the penetrating dose of TTFields to the cancer and/or to minimize hotspots or toxicity at the skull, scalp, or skin interface.

In some embodiments, a subcutaneous agent can include an osmolality of the agent in the range from about 50 mOsm/kg to about 500 mOsm/kg, optionally in the range from about 150 mOsm/kg to about 450 mOsm/kg, optionally in the range from about 250 mOsm/kg to about 350 mOsm/kg, or optionally at about 300 mOsm/kg.

In some embodiments, the method of use of a subcutaneous agent can further comprise measuring a dose of TTFields at the skull, scalp, or skin interface and/or at the tumor; and wherein the measuring is performed before or after step 2. In some embodiments, the optimizing is performed by changing at least a portion of the agent to a different agent with a different conductivity.

In some embodiments, the conductivity of a subcutaneous agent is provided such that a transducer-scalp and/or a transducer-skin interface is provided, and a conductivity of the agent is in the range from about 0.001 S/m to about 100 S/m, or in the range from about 0.001 S/m to about 50 S/m, optionally in the range from about 0.001 S/m to about 25 S/m (can be unique for individual patents). In some embodiments, the conductivity of the subcutaneous agent is optimized at a conductivity in the range from about 0.1 S/m to about 11 S/m, optionally at about 1 S/m, or at about 2 S/m, at about 3 S/m, at about 4 S/m, or at about 5 S/m, at about 6 S/m, at about 7 S/m, at about 8 S/m, at about 9 S/m, or at about 10 S/m, depending upon the specific patient and depth of treatment. In some embodiments, the conductivity values for the subcutaneous agent refer to the value measured included at least a portion of the skin. In some embodiments, the method further comprises applying a vascular endothelial growth factor (VEGF) inhibitor at or under the interface. The inhibitor can optionally comprise bevacizumab or a biosimilar, a VEGF tyrosine kinase small molecule inhibitor, sunitinib, sorafenib, vandetanib, or a combination thereof.

In the various embodiments, the electric field is an alternating electric field at a frequency in the range from about 100 kHz to about 500 kHz, or in the range from about 100 kHz to about 300 kHz,² or at about 150 kHz, or optionally at about 200 kHz. In some embodiments the frequency can be about 150 kHz for body or about 200 kHz for head treatments.

In some embodiments, the methods disclosed herein are used in combination with a chemotherapy, targeted therapy, and/or immunotherapy. In some embodiments, the methods disclosed herein are used in combination with an antibiotic, anti-inflammatory, corticosteroid, anti-allergen or hypoallergenic composition, a skin barrier, or a combination thereof.

In some embodiments, a method of investigating, diagnosing, and/or treating a disease or condition is disclosed herein, comprising any of the previously described methods. In some embodiments, a method for designing a transducer array for delivering TTFields or a device, comprising any of the methods disclosed herein is provided. In some embodiments, a method for minimizing side effects on a skin surface of a subject under administration of TTFields is provided, the method including any of the previously described methods.

In some embodiments, a kit for optimizing an application of TTFields comprising instructions including the methods disclosed herein is provided; optionally wherein the kit comprises one or more of a selection of agents with different conductivities for use with the instructions.

In some embodiments, a method of making a formulation for optimization of application of TTFields, the method comprising optimizing the formulation for a conductivity as described in any of the previously described methods. In some embodiments, the method of making can further comprise electron beam curing, thermo-reactive curing, UV curing, freeze thawing, dehydration, solvent exchange(s), rehydration, addition of particles or nanoparticles, or a combination thereof.

In some embodiments, a conductive composition for modulating tumor treating fields (TTFields) is disclosed herein, the composition comprising a gel or thickener including a conductivity; optionally wherein the composition is made by the methods described above. In some embodiments, the composition can be wherein the gel comprises a hydrogel, organogel, suspension, cream, emollient, paste, lotion, micro or nanoparticles, or a combination thereof. In some embodiments, the composition can be configured wherein the composition comprises poly (vinyl alcohol)/polyethylene glycol/graphene oxide, PEO (Polyethylene-Oxide)/PVP (polyvinylpyrrolidone), polysaccharide (natural), gum karaya (natural), polyacrylamide (synthetic polymer), alginate, titanium dioxide, zinc oxide, or a combination thereof. In some embodiments, the composition is non-hydrophilic, latex free, hypoallergenic, or a combination thereof. In some embodiments, the composition further comprises an additive, an anti-vascular endothelial growth factor, a vitamin, an osmolality adjusting agent, a penetrating agent, or a combination thereof. Osmolality adjusting agents are known in the art and can include an inert solute such as a sugar or a salt.

The technology herein can be provided in a system. System and methods for determining conductivity of a formulation at any position between a transducer array relative to a subject's treatment area, which may be used in treating cancer in the subject, are provided. These techniques may include constructing, based on one or more images, a representation of the subject's cancer that includes information for a plurality of structures including one or more tumors positioned within the subject's body, brain, or head. The representation of the subject's structures may be used to calculate electric field propagation for one or more arrangements of a transducer array on a surface of the subject and for a formulation disposed between one or more transducers and a treatment area. These techniques may further include determining one or more rate of energy absorption distributions and/or one or more electric field distributions using the calculated electric field propagation for multiple arrangements of the transducer array and one or more factors in the formulation. A rate of energy absorption distribution may indicate a rate of energy absorbed at the one or more tumors or at one or more skin areas. An electric field distribution may indicate an amount of electric field at individual regions of the subject's treatment area that include the one or more tumors. Using the one or more rate of energy absorption distributions and/or the one or more electric field distributions, an indication of how to configure a conductivity of a formulation between a transducer on the subject's skin and the subject's treatment area such that at least a portion of the one or more tumors are exposed to electric fields emitted by the transducer array may be generated. In another example, a plurality of tumors, or a plurality of treatment areas are targeted. The technology can also be applied to delocalized or general treatment areas.

Another aspect of the present invention provides a pharmaceutical composition comprising an effective amount of a composition described herein. Yet another aspect of the present invention provides a pharmaceutical composition comprising one or more other compositions described herein, wherein the pharmaceutical composition is formulated for topical administration, subcutaneous administration, parenteral administration, or administration via implanted reservoir. Provided in yet another aspect is a pharmaceutical composition, wherein the pharmaceutical composition is formulated for subcutaneous injection, intravenous injection, intraperitoneal injection, or intramuscular injection.

The methods herein can be used in combination with an administration to any cell for any purpose. In some embodiments, the cell is a cancer cell, and the cell can be in vitro or in vivo. The technology can be applied to treatments using TTFields for any cancer. Any type of cancer can be targeted by selecting the appropriate cell-targeting construct. Examples of suitable cancer types include cancers of the skin, lung, stomach, bone, esophagus, liver, testicular, lymph nodes, brain, heart, central nervous system, tongue, throat, salivary glands, colon, breast, prostate, pancreas, ovaries, uterus, endometrial tubes, as well as, leukemia, melanoma, renal cell carcinoma, multiple myeloma, and any other cancer that can be inhibited (e.g., inhibition of growth or proliferation) by a cancer treating compound.

The method is especially useful to research, treat, or inhibit cancer or a tumor in a host. Thus, the invention provides, as a related aspect, a method of treating or preventing cancer or a tumor in a mammal comprising one or more methods disclosed herein. An “anti-cancer” or “anti-tumor” effective treatment is an amount sufficient to treat or inhibit, to any degree, the onset or progression of a cancer or tumor.

The dose administered to a mammal, particularly a human, in the context of the present invention should be sufficient to elicit a therapeutic response in the mammal over a reasonable time frame. The dose will be determined by the strength of the particular compound or composition administered and the condition of the mammal (e.g., human), as well as the body weight of the mammal to be treated. The size of the dose also will be determined by the existence, nature, and extent of any adverse side effects that might accompany the administration of a particular compound or composition.

In some embodiments, a suitable concentration of a compound in pharmaceutical compositions for topical administration is 0.05 to 15% (by weight). In some embodiments, the concentration for topical administration is from 0.02 to 5%. In other embodiments, the concentration is from 0.1 to 3%. Ultimately, the attending physician will decide the dosage and the amount of the compound of the invention with which to treat each individual patient, taking into consideration a variety of factors, such as age, body weight, general health, diet, sex, compound or composition to be administered, route of administration, and severity of the disease being treated.

One skilled in the art will appreciate that suitable methods of administering a compound of the present invention or a composition thereof to a mammal such as a human, are known, and, although more than one route can be used to administer a particular composition, a particular route can provide a more immediate and more effective reaction than another route.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting. Although methods and materials similar or equivalent to those described herein can be used in the practice or testing of this disclosure, suitable methods and materials are described below.

### EXAMPLES

The invention now being generally described, it will be more readily understood by reference to the following examples which are included merely for purposes of illustration of certain aspects and embodiments of the present invention and are not intended to limit the invention.

### Example 1 TTFields Distribution Saturates with Respect to Hydrogel Electric Conductivity

Magnetic resonance imaging data sets from 7 glioblastoma patients (FIG. 1A) and attenuation-corrected positron emission tomography-computed tomography data sets from 3 non-small cell lung carcinoma (FIG. 1B) and 2 ovarian carcinoma patients (FIG. 1C) were used to fully segment various anatomic structures. A 3-dimensional finite element mesh model was generated and then solved for the distribution of applied electric fields, rate of energy deposition, and current density at the gross tumor volumes (GTVs) and clinical target volumes (CTVs). Electric field-volume histograms, specific absorption rate-volume histograms, and current density-volume histograms were generated, by which plan quality metrics were used to evaluate relative differences in field coverage between models at various hydrogel conductivities. It was found in the results that TTFields coverage at the GTV or CTV increased up to 0.5 S/m for head and 1.0 S/m for thorax and pelvis models, and no additional increase was observed after these saturation points. The scalp and skin hotspots also increased accordingly. Although the scalp hotspots increased by +4.2%, +7.5%, and +3.2% in E₅%, SAR₅%, and CD₅%, the skin hotspots increased by as much as +21.7%, +51.3%, and +41.0%, respectively. A key conclusion was that TTFields delivery for the treatment of cancer can be modulated by the conductivity of the hydrogel at the transducer-scalp or transducer-skin interface. Optimizing this aspect of TTFields delivery may increase tumor control while minimizing toxicity at the skull, scalp, or skin.

Our study revealed that there is a saturation point at which increasing the hydrogel electric conductivity beyond a certain value resulted in no further increase in TTFields coverage at the GTV. This saturation point appeared to be different in the head, thorax, and pelvis models (FIG. 1A, FIG. 1B, FIG. 1C). For the head models, the saturation point is near 0.5 S/m and increasing the hydrogel conductivity from 0.1 to 0.5 S/m resulted an average increase of +4.5% (range 2.0-9.4%) in E₉₅%, +8.6% (range 3.5-23.8%) in SAR₉₅%, and +4.4% (range 3.3-6.5%) in CD₉₅% at the GTV (FIGS. 2A-C). There was negligible change in 95% TTFields coverage beyond 0.5 S/m. Similarly, the median coverage at the GTV increased on average by +4.2% (range 2.9-6.7%) in E₅₀%, +10.2% (range 6.9-14.7%) in SAR₅₀%, and +4.5% (range 3.2-6.8%) in CD₅₀% (FIGS. 3A-C). The GTV hotspot, as denoted by E₅%, SAR₅%, and CD₅%, increased by an average of +4.6% (range 2.7-7.4%), +9.2% (range 7.5-14.5%), and +4.8% (range 1.7-11.4%), respectively (FIGS. 4A-C). In addition, the scalp hotspot, as denoted by E₅%, SAR₅%, and CD₅%, increased on average by +4.2% (range 0.6-7.2%), +7.5% (range 5.2-12.0%), and +3.2% (range 2.2-4.6%), respectively (FIGS. 5A-C). However, there was also negligible change in the median (50%) coverage of TTFields and hotspot (5%) beyond 0.5 S/m. The 95% and median (50%) coverage to the scalp followed a similar pattern of increase in intensity as a function of hydrogel electric conductivity, and exhibited saturation points around 0.5 S/m (95%, FIGS. 6A-C and 50%, FIGS. 7A-C).

For the thorax and pelvis models, the saturation point for hydrogel conductivity was observed at 1.0 S/m and increasing the hydrogel conductivity from 0.1 to 1.0 S/m yielded an average increase of +21.7% (range 2.4-41.7%) in E₉₅%, +51.3% (range 0.0-114.2%) in SAR₉₅%, and +41.0% (range 11-122.2%) in CD₉₅% at the CTV (FIGS. 2D-F). There was negligible change in 95% TTFields coverage beyond 1.0 S/m. The average increase in median coverage at the CTV, quantified by the E₅₀% metric, was +22.5% (range 3.2-38.8%) for E₅₀%, +50.4% (range 0-98.3%) for SAR₅₀%, and +24.4% (range 11.8-38.6) for CD₅₀% (FIGS. 3D-F). The CTV hotspot, as denoted by E₅%, SAR₅%, and CD₅% increased by an average of +26.6% (range 9.1-41.7%), +51.0% (range 0-114.2%), and +21.7% (range 2.4-40.7%), respectively (FIGS. 4D-F). In addition, the skin hotspot, denoted by E₅%, SAR₅%, and CD₅%, increased by +22.8% (range 9.6-38.9%), +45.7% (range 20.6-87.8%), and +20.6% (range 9.5-29.9%), respectively (FIGS. 5D-F). The 95% and median (50%) coverage to the skin also followed a similar pattern of increase in intensity as a function of hydrogel electric conductivity, although the saturation points were exhibited slightly above 1 S/m, closer to 10 S/m (FIGS. 6D-6F and FIGS. 7D-7F).

### Example 2 TTFields Distribution Varies Among Different Patient Models

We observed that each patient model possessed an individualized increase in TTFields as a function of increasing hydrogel electric conductivity (FIGS. 2-7). FIG. 1 are the associated distribution maps for the series of patients studied with their respective location of the tumor volume(s) on various cut-planes. FIGS. 2-7 are the associated volume-histograms for Electric Field, SAR, and current density of the tumor volumes for different patients, and the skin/scalp as a function of modulated electric conductivity of the applied hydrogel between TTFields emitters and the patient's skin.

Generally, the greatest percentage increase was observed in the scalp or skin rather than the targets GTV or CTV. For the head models (n=7, FIG. 1A), when the hydrogel electric conductivity was set at the nominal value of 0.1 S/m and experimental value of 0.5 S/m, the average increase in TTFields of the GTV was +4.7% in E₉₅%, +4.3% in E₅₀%, and +4.8% in E₅% (FIGS. 2A, 3A, and 4A). For the scalp, when the hydrogel conductivity was increased from 0.1 to 0.5 S/m, the average increase was +4.2% in E₉₅%, +5.6% in E₅₀%, and +4.4% in E₅% hotspot (FIGS. 6A and 7A, FIG. 5A). In contrast, the SAR exhibited larger average increases in coverage within the GTV, at +10.7% for SAR₉₅%, +10.0% for SAR₅₀%, and +9.3% for SAR₅% (FIGS. 2B, 3B, and 4B). At the scalp, the average SAR increase for SAR₉₅%, SAR₅₀%, and SAR₅% was low at +1.3%, +12.1% and +7.8%, respectively (FIGS. 6B and 7B, FIG. 5B). For current density, coverage to the GTV had average increases of +4.4% in CD₉₅%, +4.6% in CD₅₀%, and +4.4% in CD₅% hotspot when hydrogel conductivity was increased from 0.1 S/m to 0.5 S/m (FIGS. 20, 3C, and 4C). Within the scalp, the average increases were +2.1%, +5.6% and +3.1% for CD₉₅%, CD₅₀%, and CD₅%, respectively (FIGS. 6C and 7C, FIG. 5C).

A similar trend was observed in the thorax (n=3, FIG. 1B) and pelvis (n=2, FIG. 1C) models where the field intensity generally increased as a function of increasing hydrogel electric conductivity. The percentage increases in the coverage metrics were much higher than those described in the head models. Specifically, the average increases in TTFields coverage to the CTVs were +25.1%, +25.3% and +23.1% for E₉₅%, E₅₀%, and E₅%, respectively (FIGS. 2D, 3D, and 4D), while the skin experienced average increases of +19.3%, +20.9%, and +22.6% for the E₉₅%, E₅₀%, and E₅% metrics, respectively (FIGS. 6D and 7D, FIG. 5D). SAR within the CTVs increased by +51.3% in SAR₉₅%, +46.0% in SAR₅₀%, and +41.1% in SAR₅% (FIGS. 2E, 3E, and 4E). Within the skin layer of these body models, the average increase was +30.8% in SAR₉₅%, +46.2% in SAR₅₀%, and +44.5% in SAR₅% (FIGS. 6E and 7E, FIG. 5E). The CTVs had an average increase in current density also, when the hydrogel conductivity was increased from 0.1 S/m to 0.5 S/m. The CD at the CTV was increased by +28.0% in CD₉₅%, +29.3% in CD₅₀%, and +27.7% in CD₅% hotspot (FIGS. 2F, 3F, and 4F). Lastly, at the skin layer, there was an average increase of +19.7% in CD₉₅%, +20.7% in CD₅₀%, and +20.1% in CD₅% hotspot (FIGS. 6F and 7F, FIG. 5F).

The experiments have shown that the medium's electrical conductivity and mass density are more important determinants than relative permittivity. Therefore, optimizing the material properties of the medium will ensure optimal delivery of Tumor Treating Fields to the target for the treatment of cancer.

### Example 3 TTField Modulation Utilizing Whole Skin Formulations

In experiments, it was found that materials to be applied to the skin with higher electric conductivity, such as, but not limited to, titanium dioxide and zinc oxide (e.g., a sunscreen) may offer additional benefit to patients receiving Tumor Treating Fields. An enhancing agent is investigated, which can be a conductivity enhancing agent, an ultraviolet light absorbing agent, or a dye (e.g., conjugated double bonds operative to absorb in a visible region). The various agents can include titanium dioxide, zinc oxide, menthyl anthranilate, octocrylene, octyl salicylate, oxybenzone, padimate O, ecamsule, cinoxate, phenylbenzimidazole, sulisobenzone, homosalate, dioxybenzone, avobenzone, or a combination thereof.

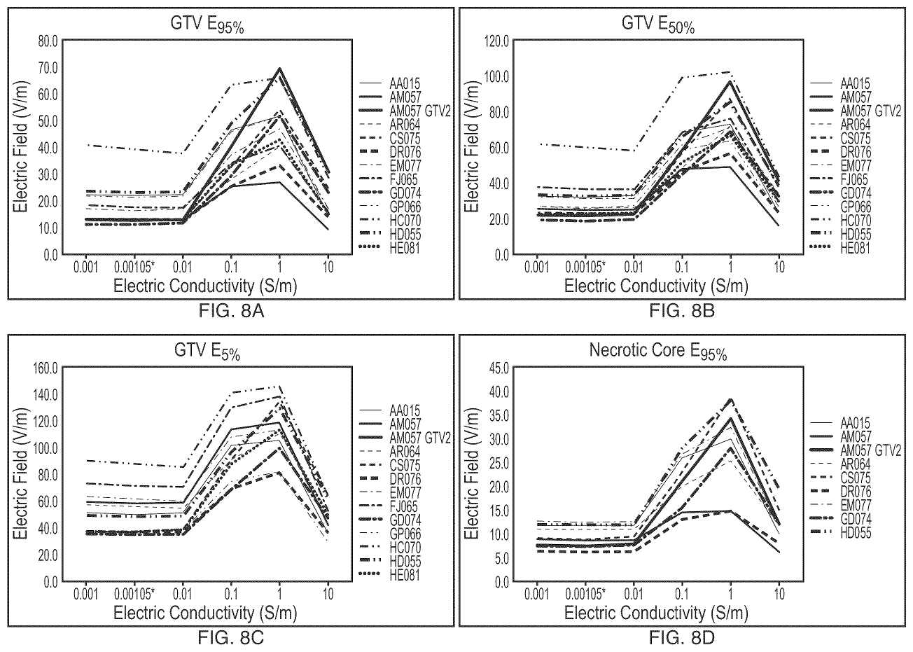

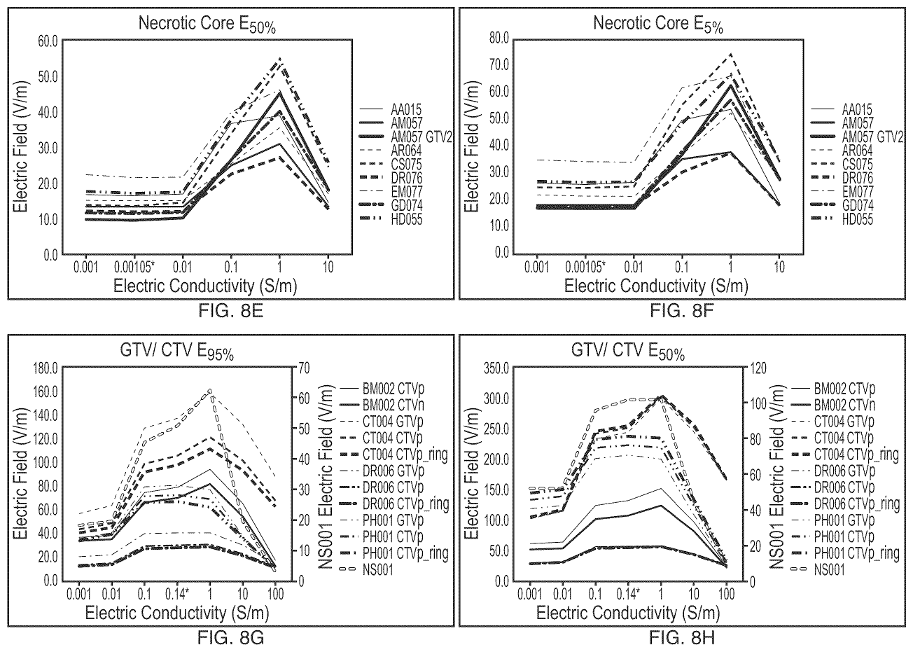

FIGS. 8-13 are the associated volume-histograms for Electric Field, SAR, and current density of the tumor volumes for different patients in our study, as a function of modulating electric conductivity of the whole skin surface/scalp surface.

FIG. 8 provides electric fields strength (E), as a function of modulating electric conductivity of the whole skin surface for head/brain, received by 95%, 50% (median), and 5% (hotspots) volume for GTV E₉₅% (FIG. 8A), GTV E₅₀% (FIG. 8B), GTV E₅% (FIG. 8C), Necrotic Core E₉₅% (FIG. 8D), Necrotic Core E₅₀% (FIG. 8E), Necrotic Core E₅% (FIG. 8F), GTV/CTV E₉₅% (FIG. 8G), GTV/CTV E₅₀% (FIG. 8H), and GTV/CTV E₅% (FIG. 8I).

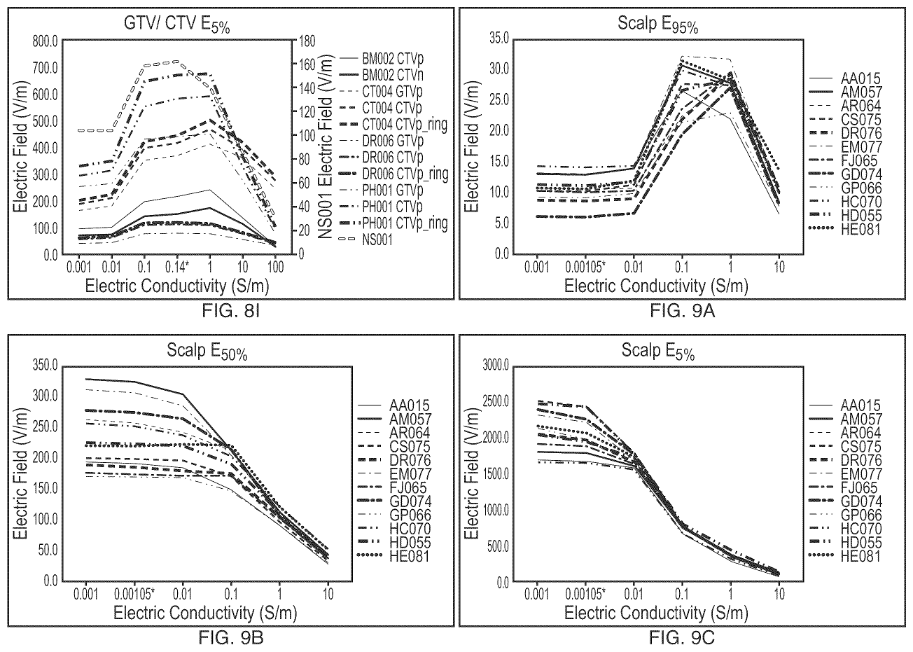

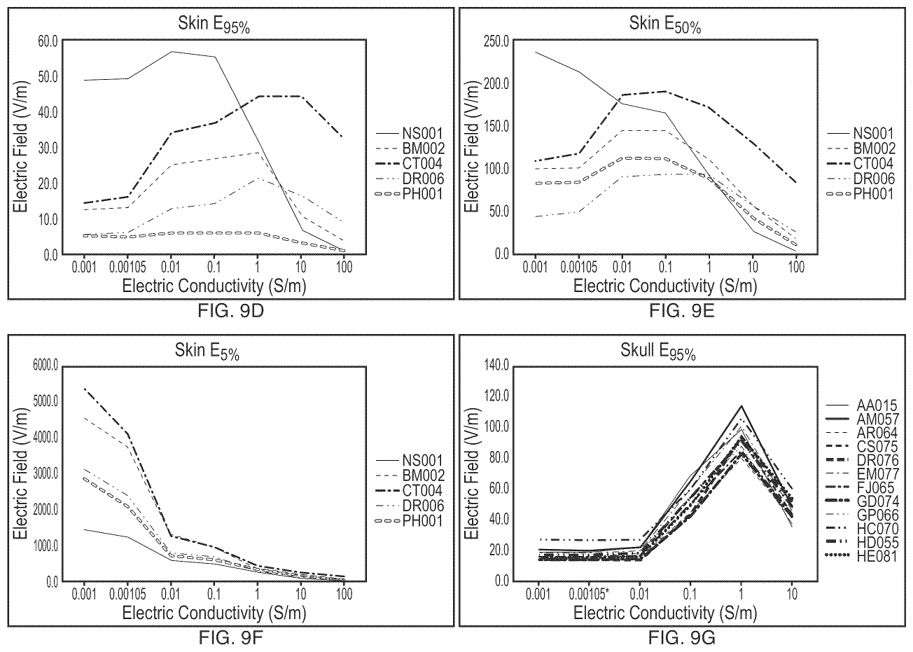

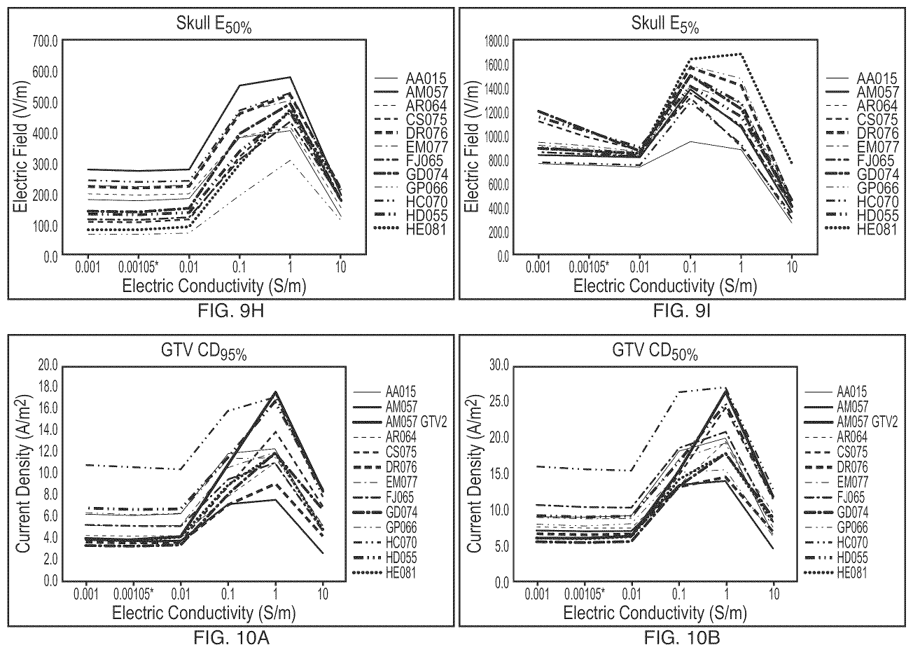

FIG. 9 provides electric fields strength (E), as a function of modulating electric conductivity of the whole skin scalp surface, for head areas received by 95%, 50% (median), and 5% (hotspots) volume for scalp E₉₅% (FIG. 9A), scalp E₅₀% (FIG. 9B), scalp E₅% (FIG. 9C), skin E₉₅% (FIG. 9D), skin E₅₀% (FIG. 9E), skin E₅% (FIG. 9F); the EVH is electric field-volume histrogram; skull E₉₅% (FIG. 9G), skull E₅₀% (FIG. 9H), and skull E₅% (FIG. 9I).

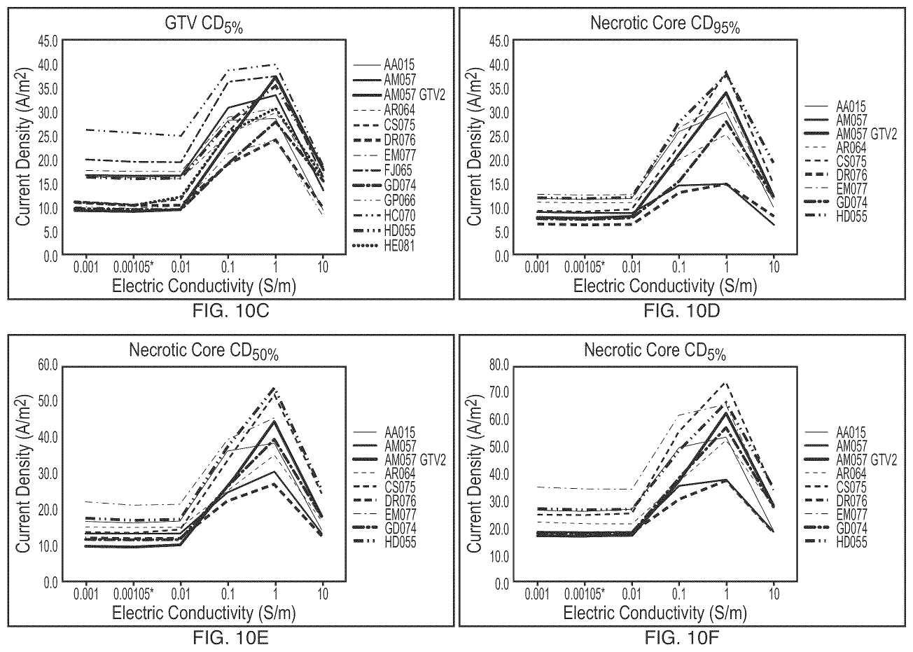

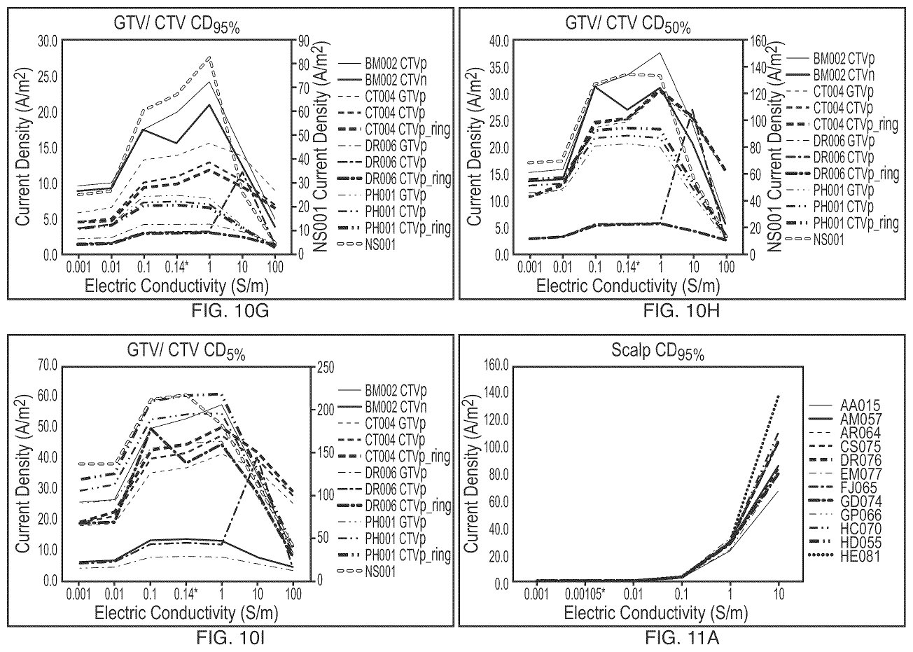

FIG. 10 provides current density (CD), as a function of modulating electric conductivity of the whole skin surface for head/brain, received by 95%, 50% (median), and 5% (hotspots) volume for GTV CD₉₅% (FIG. 10A), GTV CD₅₀% (FIG. 10B), GTV CD₅% (FIG. 10C), Necrotic Core CD₉₅% (FIG. 10D), Necrotic Core CD₅₀% (FIG. 10E), Necrotic Core CD₅% (FIG. 10F), GTV/CTV CD₉₅% (FIG. 10G), GTV/CTV CD₅₀% (FIG. 10H), and GTV/CTV CD₅% (FIG. 10I).

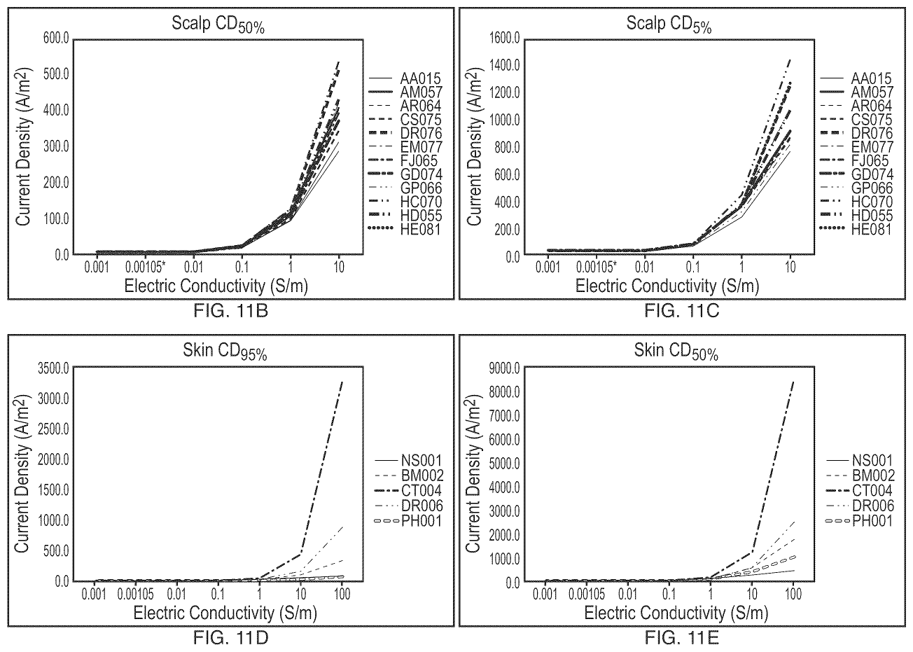

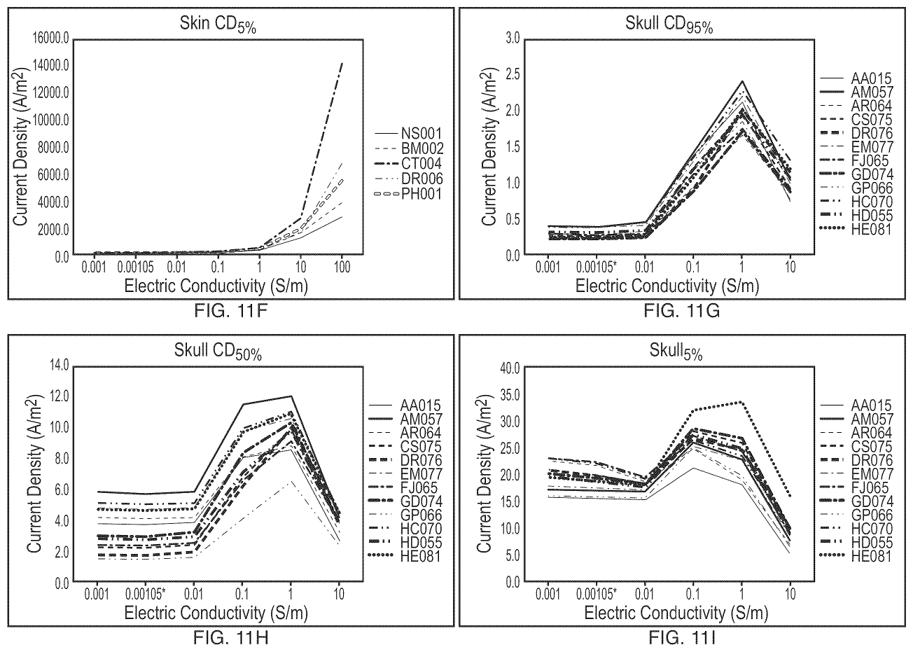

FIG. 11 provides current density (CD), as a function of modulating electric conductivity of the whole scalp surface, for head areas received by 95%, 50% (median), and 5% (hotspots) volume for scalp CD₉₅% (FIG. 11A), scalp CD₅₀% (FIG. 11B), scalp CD₅% (FIG. 11C), skin CD₉₅% (FIG. 11D), skin CD₅₀% (FIG. 11E), skin CD₅% (FIG. 11F), skull CD₉₅% (FIG. 11G), skull CD₅₀% (FIG. 11H), and skull CD₅% (FIG. 11I).

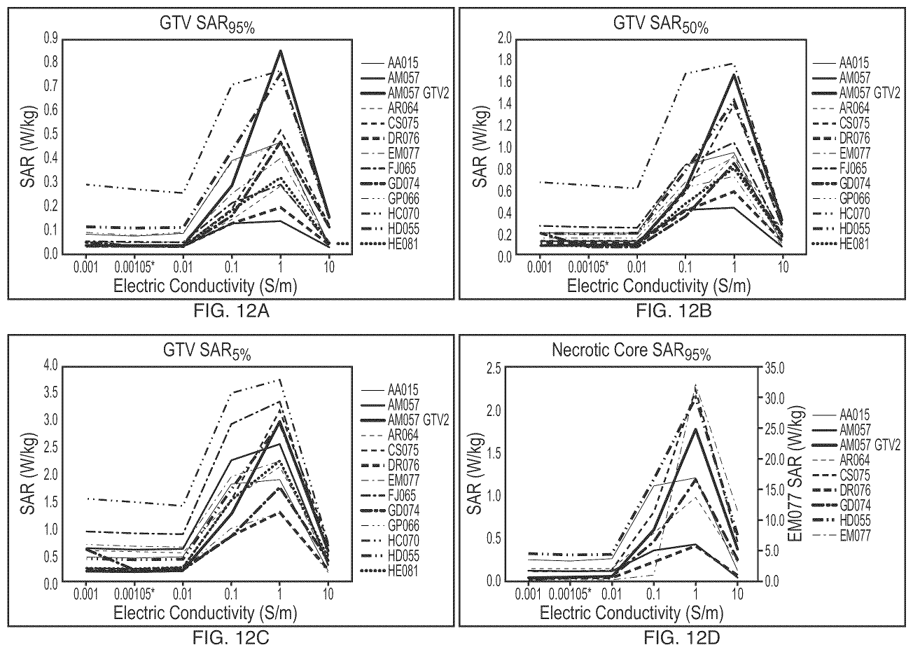

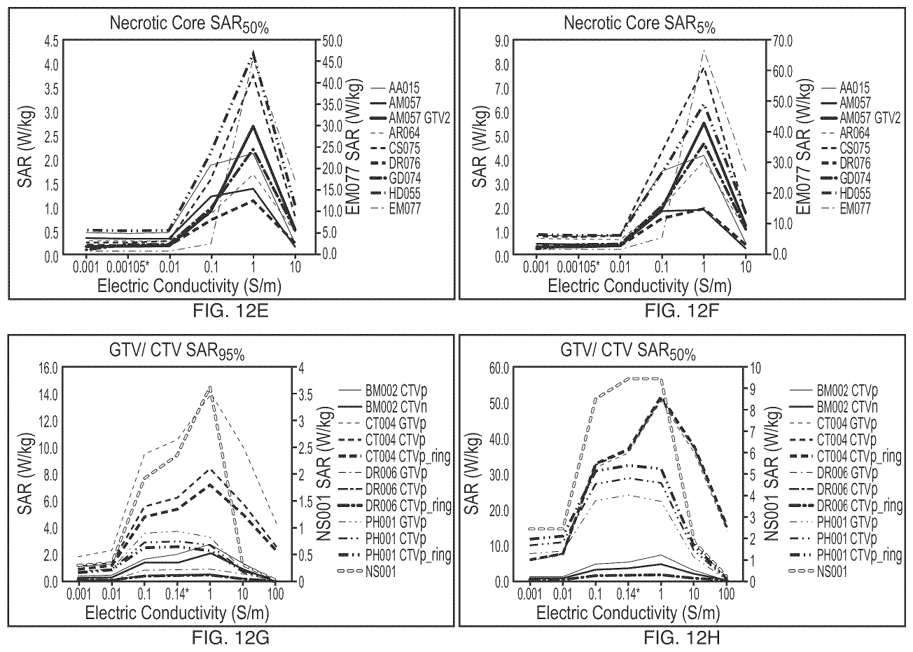

FIG. 12 provides specific absorption rate (SAR), as a function of modulating electric conductivity of the whole skin surface for head/brain, received by 95%, 50% (median), and 5% (hotspots) volume for GTV SAR₉₅% (FIG. 12A), GTV SAR₅₀% (FIG. 12B), GTV SAR₅% (FIG. 12C), Necrotic Core SAR₉₅% (FIG. 12D), Necrotic Core SAR₅₀% (FIG. 12E), Necrotic Core SAR₅% (FIG. 12F), GTV/CTV SAR₉₅% (FIG. 12G), GTV/CTV SAR₅₀% (FIG. 12H), and GTV/CTV SAR₅% (FIG. 12I).

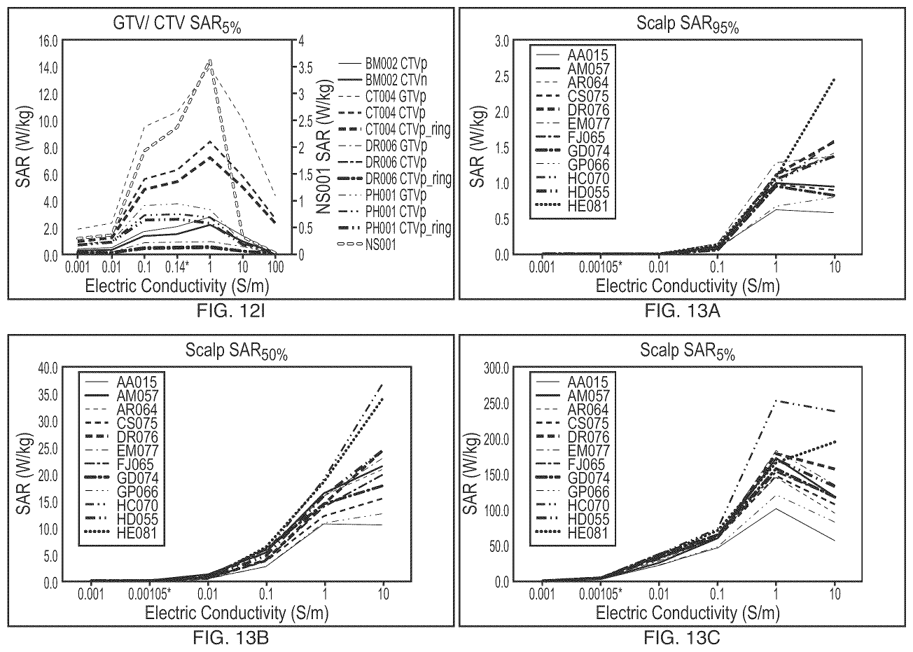

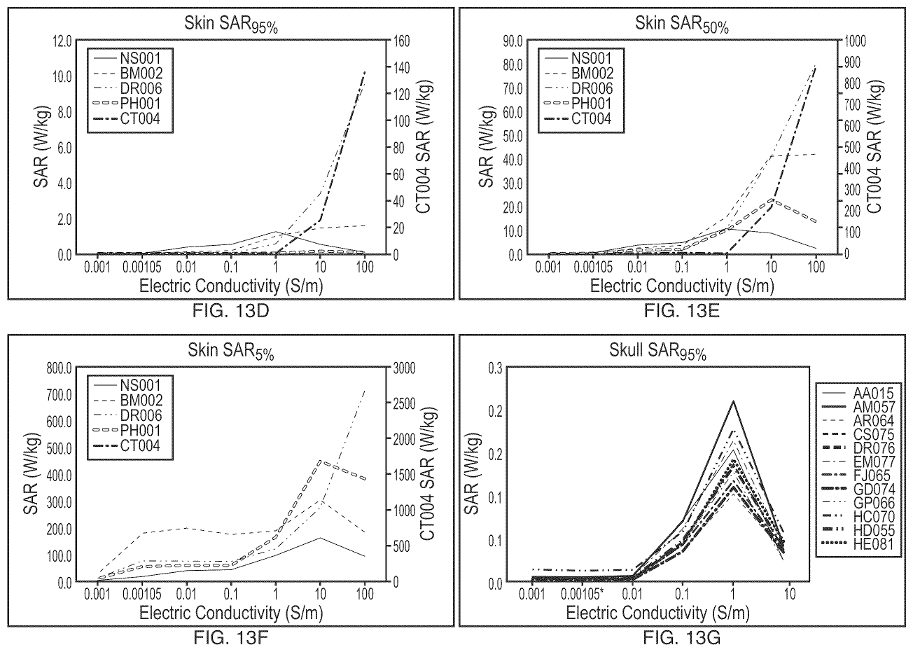

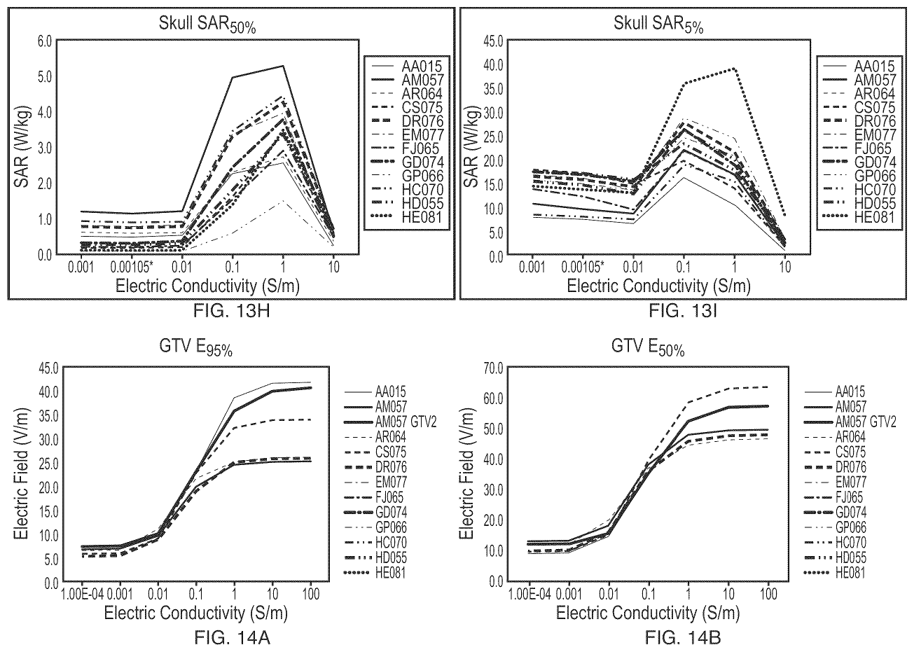

FIG. 13 provides specific absorption rate (SAR), as a function of modulating electric conductivity of the whole scalp surface, for head areas received by 95%, 50% (median), and 5% (hotspots) volume for scalp SAR₉₅% (FIG. 13A), scalp SAR₅₀% (FIG. 13B), scalp SAR₅% (FIG. 13C), skin SAR₉₅% (FIG. 13D), skin SAR₅₀% (FIG. 13E), skin SAR₅% (FIG. 13F), skull SAR₉₅% (FIG. 13G), skull SAR₅₀% (FIG. 13H), and skull SAR₅% (FIG. 13I).

### Example 4 TTField Modulation Utilizing Subcutaneous Formulations

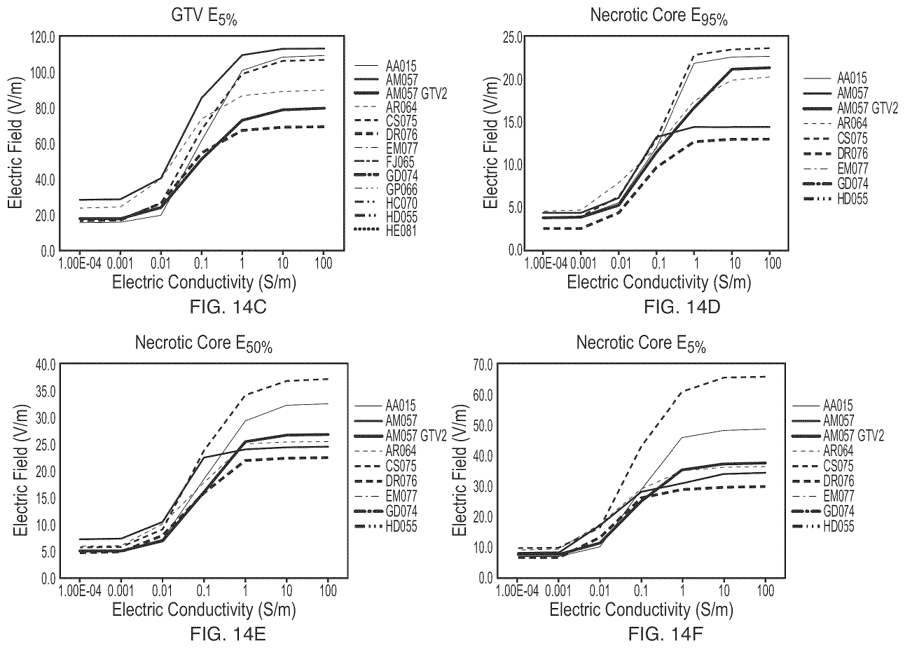

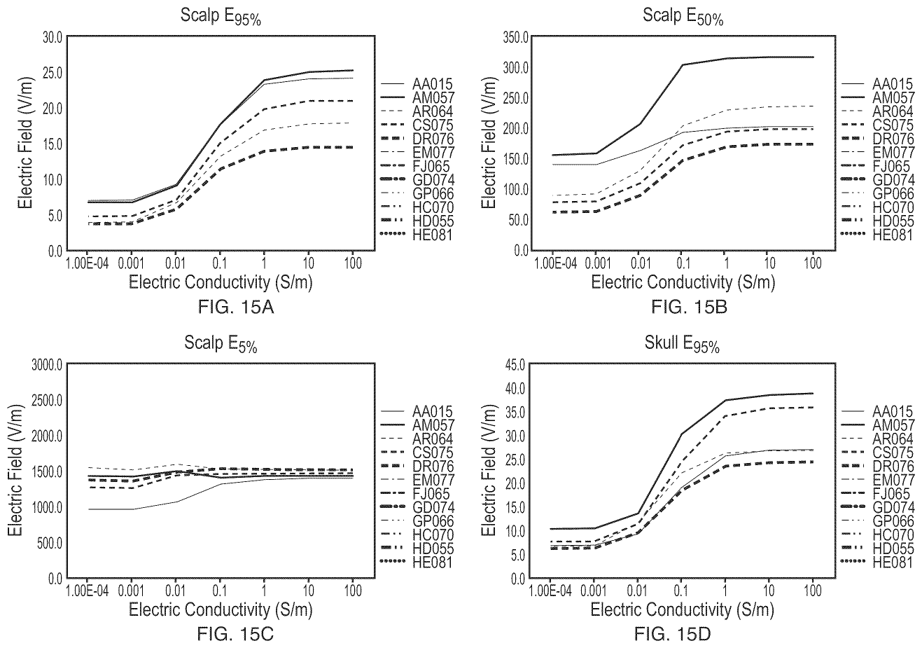

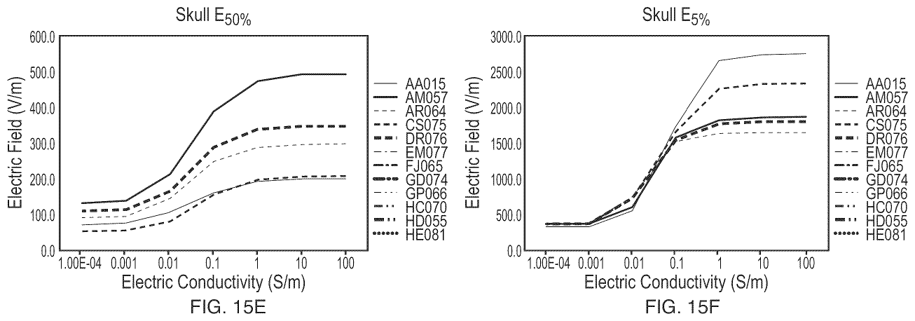

Experiments were conducted, examining a subcutaneous formulation; a barrier that can attenuate Tumor Treating Fields is the actual skin on the body surface. To potentiate the electric conductivity and charge density of skin, one can inject various formulations such as, but not limited to, gadolinium and other materials discussed above. These agents can help transduce electric fields from the skin surface to the interior of the body. FIGS. 14-15 are the associated volume-histograms for Electric Field of the tumor volumes and skin/skull, respectively, for different patients in the study, and the skin/scalp as a function of modulated electric conductivity of the injected formulation subcutaneously within the patients' skin.

FIG. 14 provides electric fields strength (E), with function of varying electric conductivity of a subcutaneous formulation (e.g., injected formulation), received by 95%, 50% (median), and 5% (hotspots) volume for GTV E₉₅% (FIG. 14A), GTV E₅₀% (FIG. 14B), GTV E₅% (FIG. 14C), Necrotic Core E₉₅% (FIG. 14D), Necrotic Core E₅₀% (FIG. 14E), and Necrotic Core E₅% (FIG. 14F).

FIG. 15 provides electric fields strength (E), with function of varying electric conductivity of a subcutaneous formulation (e.g., injected formulation) for head areas (e.g., scalp/skull) received by 95%, 50% (median), and 5% (hotspots) volume for scalp E₉₅% (FIG. 15A), scalp E₅₀% (FIG. 15B), scalp E₅% (FIG. 15C), skull E₉₅% (FIG. 15D), skull E₅₀% (FIG. 15E), and skull E₅% (FIG. 15F).

### Example 5 TTFields Distribution Differences Between Head and Body

We next compared the extent of changes in PQM metrics between the head and body models when the hydrogel conductivity was increased from 0.1 to 0.5 S/m (see Table 1). For GTVs in the head models, this resulted in a negligible increase in standard deviation of <10% in E₉₅%, E₅₀%, E₅%, SAR₅₀%, SAR₅%, CD₉₅%, CD₅₀%, and CD₅%, except for SAR₉₅% which had an increase of 34.3%. However, for the CTVs in the thorax and pelvis models, there was a greater increase in standard deviation ranging from 13.4-32.0% across all of the PQM metrics. For scalp in the head models, increasing the hydrogel conductivity resulted in a negligible increase in standard deviation of <10% in E₉₅%, E₅₀%, SAR₉₅%, CD₉₅%, CD₅₀%, and CD₅%, except for E₅%, SAR₅₀%, and SAR₅%, which were increased by 17.4%, 10.7%, and 18.2%, respectively. In contrast, the thorax and pelvis models had a much greater increase in standard deviation ranging from 21.1% up to 54.7% across all of the PQM metrics.

TABLE 1
Head vs Body PQM STD:

| | PQM | Head Model Standard Deviations | | | Body Model Standard Deviations | | |
|---|---|---|---|---|---|---|---|
| | Metric | σ = 0.1 S/m | σ = 0.5 S/m | % increase | σ =0.1 S/m | σ = 0.5 S/m | % increase |
| GTV | E_{95%} | 3.2 | 3.4 | 7.4 | 34.2 | 42.8 | 25.0 |
| or | E_{50%} | 5.2 | 5.6 | 7.6 | 76.4 | 90.1 | 17.9 |
| CTV | E_{5%} | 12.7 | 13.6 | 7.5 | 125.9 | 142.8 | 13.4 |
| (Head | SAR_{95%} | 0.0 | 0.0 | 34.3 | 2.0 | 2.7 | 32.0 |
| or | SAR_{50%} | 0.1 | 0.1 | 8.6 | 12.5 | 16.1 | 28.4 |
| Body) | SAR_{5%} | 0.2 | 0.3 | 9.9 | 33.6 | 41.3 | 23.1 |
| | CD_{95%} | 0.9 | 0.9 | 3.2 | 22.1 | 27.3 | 23.5 |
| | CD_{50%} | 1.5 | 1.6 | 7.4 | 44.8 | 58.1 | 29.6 |
| | CD_{5%} | 3.6 | 3.6 | 0.1 | 72.2 | 92.0 | 27.4 |
| Scalp | E_{95%} | 1.6 | 1.6 | 1.0 | 15.6 | 19.0 | 21.5 |
| or | E_{50%} | 57.6 | 60.5 | 5.1 | 37.8 | 46.8 | 23.7 |
| Skin | E_{5%} | 259.2 | 304.2 | 17.4 | 183.4 | 255.1 | 39.1 |
| (Head | SAR_{95%} | 0.0 | 0.0 | 2.6 | 0.2 | 0.3 | 37.3 |
| or | SAR_{50%} | 0.0 | 0.0 | 10.7 | 1.9 | 2.6 | 41.2 |
| Body) | SAR_{5%} | 1.0 | 1.2 | 18.2 | 53.2 | 82.3 | 54.7 |
| | CD_{95%} | 0.0 | 0.0 | 4.8 | 2.2 | 2.7 | 21.2 |
| | CD_{50%} | 0.7 | 0.7 | 5.6 | 5.3 | 6.5 | 22.2 |
| | CD_{5%} | 2.5 | 2.5 | 1.3 | 27.3 | 33.1 | 21.1 |

³⁶ CURRENT PROTOCOLS IN MOLECULAR BIOLOGY (CPMB), (2014). Frederick M. Ausubel (ed.), John Wiley and Sons (ISBN 047150338X, 9780471503385).
³⁷ CURRENT PROTOCOLS IN PROTEIN SCIENCE (CPPS), (2005). John E. Coligan (ed.), John Wiley and Sons, Inc.
³⁸ CURRENT PROTOCOLS IN IMMUNOLOGY (CPI) (2003). John E. Coligan, ADA M Kruisbeek, David H Margulies, Ethan M Shevach, Warren Strobe, (eds.) John Wiley and Sons, Inc. (ISBN 0471142735, 9780471142737).

### REFERENCES

¹Usach, I., Martinez, R., Festini, T. et al. Subcutaneous Injection of Drugs: Literature Review of Factors Influencing Pain Sensation at the Injection Site. Adv Ther 36, 2986-2996 (2019).
²Kassubek R, Mathieu R. First Report of Tumor Treating Fields (TTFields) Therapy for Glioblastoma in Comorbidity with Multiple Sclerosis. Brain Sci. 2022 Apr. 13; 12(4): 499. doi: 10.3390/brainsci12040499. PMID: 35448029; PMCID: PMC9032427.
³Stupp R, Taillibert S, Kanner A, et al. Effect of Tumor-Treating Fields plus maintenance temozolomide vs maintenance temozolomide alone on survival in patients with glioblastoma. JAMA. 2017; 318(23): p. 2306-2316.
⁴Stupp R, Wong E T, Kanner A A, et al. NovoTTF-100A versus physician's choice chemotherapy in recurrent glioblastoma: a radomised phase III study of a novel treatment modality. Eur J Cancer. 2012; 48(14): p. 2192-2202.
⁵ Gera N, Yang A, Holtzman T S, Lee S X, Wong E T, Swanson K D. Tumor treating fields perturb the localization of septins and cause aberrant mitotic exit. PLOS One. 2015; 10(5): p. e0125269.
⁶Karanam N K, Srinivasan K, Ding L, Sishc B, Saha D, Story M D. Tumor-treating fields elicit a conditional vulnerability to ionizing radiation via the downregulation of BRCA1 signaling and reduced DNA double-strand break repair capacity in non-small cell lung cancer cell lines. Cell Death Dis. 2017; 8(3): p. e2711.
⁷Shteingauz A, Porat Y, Voloshin T, Schneiderman R S, Munster M, Zeevi E, et al. AMPK-dependent autophagy upregulation serves as a survival mechanism in response to Tumor Treating Fields (TTFields). Cell Death Dis. 2018; 9(11): p. 1074.
⁸Voloshin T, Kaynan N, Davidi S, Porat Y, Shteingauz A, Schneiderman R S, et al. Tumor-treating fields (TTFields) induce immunogenic cell death resulting in enhanced antitumor efficacy when combined with anti-PD-1 therapy. Cancer Immunol Immunother. 2020; 69(7): p. 1191-1204.
⁹Swanson K D, Lok E, Wong E T. An overview of alternating electric fields therapy (NovoTTF therapy) for the treatment of malignant glioma. Curr Neurol Neurosci Rep. 2016; 16: p. 8.
¹⁰Toms S A, Kim C Y, Nicholas G, Ram Z. Increased compliance with tumor treating fields therapy is prognostic for improved survival in the treatment of glioblastoma: a subgroup analysis of the EF-14 phase III trial. J Neurooncol. 2019; 141(2): p. 467-473.
¹¹Ceresoli G L, Aerts J G, Dziadziuszko R, Ramlau R, Cedres S, van Meerbeeck J P, et al. Tumour Treating Fields in combination with pemetrexed and cisplatin or carboplatin as first-line treatment for unresectable malignant pleural mesothelioma (STELLAR): a multicentre, single-arm phase 2 trial. Lancet Oncol. 2019; 20(12): p. 1702-1709.
¹²Lacouture M E, Anadkat M J, Ballo M T, Iwamoto F, Jeyapalan S A, La Rocca R V, et al. Prevention and Management of Dermatologic Adverse Events Associated With Tumor Treating Fields in Patients With Glioblastoma. Frontiers in Oncology. 2020; 10(1045).
¹³Lacouture M E, Davis M E, Elzinga G, Butowski N, Tran D, Villano J L, et al. Characterization and Management of Dermatologic Adverse Events With the NovoTTF-100A System, a Novel Anti-mitotic Electric Field Device for the Treatment of Recurrent Glioblastoma. Seminars in Oncology. 2014; 41: p. S1-S14.
¹⁴Lok E, San P, Hua V, Phung M, Wong E T. Analysis of physical characteristics of Tumor Treating Fields for human glioblastoma. Cancer Med. 2017; 6(6): p. 1286-1300.
¹⁵Lok E, San P, White V, Liang O, Widick P C, Reddy S P, et al. Tumor treating Fields for ovarian carcinoma: a modeling study. Adv Radiat Oncol. 2021; 6(4): p. 100716.
¹⁶Timmons J J, Lok E, San, Bui K, Wong E T. End-to-end workflow for finite element analysis of tumor treating fields in glioblastomas. Phys Med Biol. 2017; 62(21): p. 8264-8282.
¹⁷Wenger C, Salvador R, Basser P J, Miranda P C. The electric field distribution in the brain during TTFields therapy and its dependence on tissue dielectric properties and anatomy: a computational study. Physics in Medicine and Biology. 2016; 60(18): p. 7339-7357.
¹⁸Kirson E D, Dbaly V, Tovarys F, Vymazal J, Soustiel J F, Itzhaki A, et al. Alternating electric fields arrest cell proliferation in animal tumor models and human brain tumors. Proc Natl Acad Sci USA. 2007; 104(24): p. 10152-10157.
¹⁹Dressel M, Scheffler M. Verifying the Drude response. Ann. Phys. 2006; 15(7-8): p. 535-544.
²⁰fda.gov/cdrh_docs/pdf18/H180002D.pdf.
²¹. Wenger C, Salvador R, Basser P J, Miranda P C. The electric field distribution in the brain during TTFields therapy and its dependence on tissue dielectric properties and anatomy: a computational study. Phys Med Biol. 2015; 60(18): p. 7339-7357.
²²Lok E, Hua V, Wong E T. Computed modeling of alternating electric fields therapy for recurrent glioblastoma. Cancer Med. 2015; 4(11): p. 1697-1699.
²³Yano K, Brown L F, Detmar M. Control of hair growth and follicle size by VEGF-mediated angiogenesis. J Clin Invest. 2001; 107(4): p. 409-417.
²⁴Casak S J, Lemery S J, Chung J, Fuchs C, Schrieber S J, Chow E C, et al. FDA's Approval of the First Biosimilar to Bevacizumab. Clin Cancer Res. 2018; 24(18): p. 4365-4370.
²⁵Morabito A, De Maio E, Di Maio M, Normanno N, Perrone F. Tyrosine kinase inhibitors of vascular endothelial growth factor receptors in clinical trials: current status and future directions. Oncologist. 2006; 11(7): p. 753-764.
²⁶Isselbacher, et al. (1996). HARRISON'S PRINCIPLES OF INTERNAL MEDICINE, 13 ed., 1814-1882.
²⁷THE MERCK MANUAL OF DIAGNOSIS AND THERAPY, (2011). 19^{th} Edition, published by Merck Sharp & Dohme Corp., (ISBN 978-0-911910-19-3).
²⁸THE ENCYCLOPEDIA OF MOLECULAR CELL BIOLOGY AND MOLECULAR MEDICINE, Robert S. Porter et al. (eds.), published by Blackwell Science Ltd., 1999-2012 (ISBN 9783527600908).
²⁹MOLECULAR BIOLOGY AND BIOTECHNOLOGY: A COMPREHENSIVE DESK REFERENCE, (1995). Robert A. Meyers (ed.), published by VCH Publishers, Inc. (ISBN 1-56081-569-8).
³⁰IMMUNOLOGY, (2006). Werner Luttmann, published by Elsevier.
³¹JANEWAY'S IMMUNOBIOLOGY, (2014). Kenneth Murphy, Allan Mowat, Casey Weaver (eds.), Taylor & Francis Limited, (ISBN 0815345305, 9780815345305).
³²LEWIN'S GENES X I, (2014). published by Jones & Bartlett Publishers (ISBN-1449659055).
³³Michael Richard Green and Joseph Sambrook, (2012). MOLECULAR CLONING: A LABORATORY MANUAL, 4th ed., Cold Spring Harbor Laboratory Press, Cold Spring Harbor, N.Y., USA (ISBN 1936113414).
³⁴Davis et al., (2012). BASIC METHODS IN MOLECULAR BIOLOGY, Elsevier Science Publishing, Inc., New York, USA (ISBN 044460149X).
³⁵LABORATORY METHODS IN ENZYMOLOGY: DNA, (2013). Jon Lorsch (ed.) Elsevier (ISBN 0124199542).
³⁶CURRENT PROTOCOLS IN MOLECULAR BIOLOGY (CPMB), (2014). Frederick M. Ausubel (ed.), John Wiley and Sons (ISBN 047150338X, 9780471503385).
³⁷CURRENT PROTOCOLS IN PROTEIN SCIENCE (CPPS), (2005). John E. Coligan (ed.), John Wiley and Sons, Inc.
³⁸CURRENT PROTOCOLS IN IMMUNOLOGY (CPI) (2003). John E. Coligan, ADA M Kruisbeek, David H Margulies, Ethan M Shevach, Warren Strobe, (eds.) John Wiley and Sons, Inc. (ISBN 0471142735, 9780471 142737).

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The foregoing written specification is considered to be sufficient to enable one skilled in the art to practice the present aspects and embodiments. The present aspects and embodiments are not to be limited in scope by examples provided, since the examples are intended as a single illustration of one aspect and other functionally equivalent embodiments are within the scope of the disclosure. Various modifications in addition to those shown and described herein will become apparent to those skilled in the art from the foregoing description and fall within the scope of the appended claims. The advantages and objects described herein are not necessarily encompassed by each embodiment. Those skilled in the art will recognize, or be able to ascertain using no more than routine experimentation, many equivalents to the specific embodiments described herein. Such equivalents are intended to be encompassed by the following claims.

What is claimed is:

1. A method for modulating tumor treating fields (TTFields) for the treatment of cancer, the method comprising the steps of:
(1) obtaining a topical agent disposed between a TTField transducer and a subject's skin; the agent operative to provide a transducer-scalp or a transducer-skin interface between the transducer, agent, and/or the skin; and
(2) optimizing a conductivity of the agent to increase the penetrating dose of TTFields to the cancer and/or to minimize hotspots or toxicity at the skull, scalp, or skin interface.

2. The method of claim 1, wherein the optimizing minimizes TTField's intensity (charge carrier density) on the skull, scalp, or skin while maximizing field intensity (charge carrier density) on a tumor.

3. The method of claim 1, further comprising measuring a dose of TTFields at the skull, scalp, or skin interface and/or at the tumor; and wherein the measuring is performed before or after step 2.

4. The method of claim 1, wherein the optimizing is performed by changing at least a portion of the agent to a different agent with a different conductivity.

5. The method of any preceding claim, wherein the agent comprises a composition, wherein the composition includes a hydrogel, organogel, suspension, cream, emollient, paste, lotion, lipid, micro or nanoparticles, or a combination thereof.

6. The method of claim 1, wherein the agent is in the form of an organogel, a hydrogel, a suspension, cream, emollient, paste, lotion, adhesive, lipid, micro or nanoparticles, or a combination thereof, removably affixed to the TTField transducer.

7. The method of claim 6, wherein the agent is configured to be peeled off by a healthcare provider such that a different agent can be applied to the transducer.

8. The method of any preceding claim, wherein a transducer-scalp interface is provided, and a conductivity of the agent is in the range from about 0.001 S/m to about 10 S/m or wherein a transducer-skin interface is provided and a conductivity of the agent is in the range from about 0.001 S/m to about 100 S/m.

9. The method of any preceding claim, further comprising applying a vascular endothelial growth factor (VEGF) inhibitor at or under the interface.

10. The method of claim 9, wherein the inhibitor comprises, bevacizumab or a biosimilar, a VEGF tyrosine kinase small molecule inhibitor, sunitinib, sorafenib, vandetanib, or a combination thereof.

11. The method of any preceding claim, wherein the optimized agent is in place for a time period in the range from about 18 hours or more per day, continuously.

12. The method of any preceding claim, wherein the topical agent is operative to penetrate at least a portion of the skin and to change the conductivity of the skin to a value in the range from about 0.001 S/m to about 100 S/m.

13. The method of claim 12, wherein the agent comprises a conductive agent comprising titanium dioxide, zinc oxide, and/or a hydrogel, organogel, suspension, cream, emollient, paste, lotion, micro or nanoparticles, poly (vinyl alcohol)/polyethylene glycol/graphene oxide, hyaluronic acid, dimethyl sulfoxide, PEO (Polyethylene-Oxide)/PVP (polyvinylpyrrolidone), polysaccharide (natural), gum karaya (natural), polyacrylamide (synthetic polymer), alginate, menthyl anthranilate, octocrylene, octyl salicylate, oxybenzone, padimate O, ecamsule, cinoxate, dimethyl sulfoxide, phenylbenzimidazole, sulisobenzone, homosalate, dioxybenzone, avobenzone, a dye, an ultraviolet absorbing agent, or a combination thereof.

14. A method for modulating tumor treating fields (TTFields) for the treatment of cancer, the method comprising the steps of:
(1) administering a subcutaneous agent to an area disposed under a subject's skin, which is further under a TTField transducer; the agent operative to provide a conductivity under a transducer-scalp or a transducer-skin interface between the transducer, agent, and/or skin; and
(2) optimizing a conductivity of the agent to increase the penetrating dose of TTFields to the cancer and/or to minimize hotspots or toxicity at the skull, scalp, or skin interface.

15. The method of claim 14, wherein the osmolality of the agent is in the range from about 50 mOsm/kg to about 500 mOsm/kg, optionally at about 300 mOsm/kg.

16. The method of any one of claims 14-15, further comprising measuring a dose of TTFields at the skull, scalp, or skin interface and/or at the tumor; and wherein the measuring is performed before or after step 2.

17. The method of claim 14, wherein the optimizing is performed by changing at least a portion of the agent to a different agent with a different conductivity.

18. The method of claim 14, wherein a transducer-scalp and/or a transducer-skin interface is provided and a conductivity of the agent is in the range from about 0.001 S/m to about 100 S/m.

19. The method of claim 14, further comprising applying a vascular endothelial growth factor (VEGF) inhibitor at or under the interface.

20. The method of claim 19, wherein the inhibitor comprises, bevacizumab or a biosimilar, a VEGF tyrosine kinase small molecule inhibitor, sunitinib, sorafenib, vandetanib, or a combination thereof.

21. The method of any preceding claim, wherein the electric field is an alternating electric field at a frequency in the range from about 100 kHz to about 500 kHz, or about 150 kHz clinically used for body and about 200 kHz used for brain.

22. The method of any preceding claim, wherein the method is used in combination with a surgical procedure, radiotherapy, chemotherapy, targeted therapy, and/or immunotherapy.

23. The method of any preceding claim, wherein the method is used in combination with an antibiotic, anti-inflammatory, corticosteroid, anti-allergen or hypoallergenic composition, a skin barrier, or a combination thereof.

24. A method of investigating, diagnosing, and/or treating a disease or condition comprising the method of any preceding claim.

25. A method for designing a transducer array for delivering TTFields or a device, comprising the method of any preceding claim.

26. A method for minimizing side effects on a skin surface of a subject under administration of TTFields, the method comprising the method of any preceding claim.

27. A kit for optimizing an application of TTFields comprising instructions including the method of any preceding claim; optionally wherein the kit comprises one or more of a selection of agents with different conductivities for use with the instructions.

28. A method of making a formulation for optimization of application of TTFields, the method comprising optimizing the formulation for a conductivity as described in the method of any preceding claim.

29. The method of claim 28, further comprising electron beam curing, thermo-reactive curing, UV curing, freeze thawing, dehydration, solvent exchange(s), rehydration, addition of particles or nanoparticles, or a combination thereof.

30. A conductive composition for modulating tumor treating fields (TTFields), the composition comprising a gel or thickener including a conductivity; optionally wherein the composition is made by the method of claim 28.

31. The composition of claim 30, wherein the gel comprises a hydrogel, organogel, suspension, cream, emollient, paste, lotion, micro or nanoparticles, or a combination thereof.

32. The composition of any one of claims 30-31, wherein the composition comprises poly (vinyl alcohol)/polyethylene glycol/graphene oxide, hyaluronic acid, dimethyl sulfoxide, PEO (Polyethylene-Oxide)/PVP (polyvinylpyrrolidone), polysaccharide (natural), gum karaya (natural), polyacrylamide (synthetic polymer), alginate, titanium dioxide, zinc oxide, or a combination thereof.

33. The composition of any one of claims 30-32, wherein the composition is non-hydrophilic, latex free, hypoallergenic, or a combination thereof.

34. The composition of any one of claims 30-33, further comprising an additive, an anti-vascular endothelial growth factor, a vitamin, an osmolality adjusting agent, a penetrating agent, or a combination thereof.
